SEQUENCE LISTING (45 sequences)

SEQ ID NO: 1 - DNA, 30 bp, synthetic construct
  misc_feature 1..18: note = DNA
  misc_feature 19: note = RNA
  misc_feature 20..30: note = DNA
  source 1..30: mol_type = other DNA
cttcttccttggaactctctgacctgctgc

SEQ ID NO: 2 - DNA, 73 bp, synthetic construct
  misc_feature 1..42: note = DNA
  misc_feature 43: note = RNA
  misc_feature 44..73: note = DNA
  source 1..73: mol_type = other DNA
  misc_feature 36^37: note = three carbon spacer
ctatatatatatatatattggaccccaaaaaaaaccggtccattgtaaataaaagagttccaaggaagaaggg

SEQ ID NO: 3 - DNA, 30 bp, synthetic construct; identical to SEQ ID NO: 1
  source 1..30: mol_type = other DNA

SEQ ID NO: 4 - DNA, 17 bp, synthetic construct
  source 1..17: mol_type = other DNA
tgggcgtccagcatctc

SEQ ID NO: 5 - DNA, 21 bp, synthetic construct
  source 1..21: mol_type = other DNA
caaaggcatggagcatctgta

SEQ ID NO: 6 - DNA, 167 bp, synthetic construct
  source 1..167: mol_type = other DNA
ttctgtgtcttcccacctacagtaacaaaggcatggagcatctgtacagcatgaagtgcaagaacgtggtgcccctctctgacctgctgctggagatgctggacgcccaccgcctacatgcgcccactagccgtggaggggcatccgtggaggagacggaccaaagc

SEQ ID NO: 7 - DNA, 29 bp, synthetic construct
  source 1..29: mol_type = other DNA
ccactctctctctgatagtcagaggtgac

SEQ ID NO: 8 - DNA, 16 bp, synthetic construct
  source 1..16: mol_type = other DNA
ccactctctctctgat

SEQ ID NO: 9 - DNA, 24 bp, synthetic construct
  source 1..24: mol_type = other DNA
ggctctcatggaatggctaaagac

SEQ ID NO: 10 - DNA, 19 bp, synthetic construct
  source 1..19: mol_type = other DNA
acgctgcagtcctcgctca

SEQ ID NO: 11 - DNA, 127 bp, synthetic construct
  source 1..127: mol_type = other DNA
acagatcttgaggctctcatggaatggctaaagacaagaccaatcctgtcacctctgactaaggggattttaggatttgtgttcacgttcaccgtgcccagtgagcgaggactgcagcgtagacgct

SEQ ID NO: 12 - DNA, 24 bp, synthetic construct
  source 1..24: mol_type = other DNA
gaacacaaatcctaaaatcccctt

SEQ ID NO: 13 - DNA, 32 bp, synthetic construct
  source 1..32: mol_type = other DNA
ccactctctctctgatgaaactcacgatagaa

SEQ ID NO: 14 - DNA, 16 bp, synthetic construct
  source 1..16: mol_type = other DNA
ccactctctctctgat

SEQ ID NO: 15 - DNA, 24 bp, synthetic construct
  source 1..24: mol_type = other DNA
atccatctctacttgaaatcacag

SEQ ID NO: 16 - DNA, 23 bp, synthetic construct
  source 1..23: mol_type = other DNA
aagatccacttttttaggcagat

SEQ ID NO: 17 - DNA, 140 bp, synthetic construct
  source 1..140: mol_type = other DNA
gatggttaaactattataaacttaatacttatccatctctacttgaaatcacagaaaatgatttgattattttatcaggattgcggttctatcgtgagtttcatctgcctaaaaaagtggatcttgaaatgataataaat

SEQ ID NO: 18 - DNA, 63 bp, synthetic construct
  misc_feature 1..48: note = DNA
  misc_feature 46: note = RNA
  misc_feature 47..63: note = DNA
  source 1..63: mol_type = other DNA
  misc_feature 33^34: note = three carbon spacer
  modified_base 1: mod_base = OTHER; note = iso deoxycytosine
cccctccctcctccttcctcctgagtgccaaaaaaaacccactcaatatcagagagagagtgg

SEQ ID NO: 19 - DNA, 38 bp, synthetic construct
  misc_feature 1..19: note = DNA
  misc_feature 20: note = RNA
  misc_feature 21..38: note = DNA
  source 1..38: mol_type = other DNA
cctcactcacacctaaaatccccttagtcagaggtgac

SEQ ID NO: 20 - DNA, 19 bp, synthetic construct
  source 1..19: mol_type = other DNA
cctcactcacacctaaaat

SEQ ID NO: 21 - DNA, 34 bp, synthetic construct
  misc_feature 1..12: note = DNA
  misc_feature 13: note = RNA
  misc_feature 14..34: note = DNA
  source 1..34: mol_type = other DNA
gtgcarggcaccgacattttaggtgtgagtgagg

SEQ ID NO: 22 - DNA, 34 bp, synthetic construct
  misc_feature 1..18: note = DNA
  misc_feature 19: note = RNA
  misc_feature 20..34: note = DNA
  source 1..34: mol_type = other DNA
caccgtcgtagatgaaactcacgatagaaccgca

SEQ ID NO: 23 - DNA, 18 bp, synthetic construct
  source 1..18: mol_type = other DNA
caccgtcgtagatgaaac

SEQ ID NO: 24 - DNA, 33 bp, synthetic construct
  misc_feature 1..12: note = DNA
  misc_feature 13: note = RNA
  misc_feature 14..33: note = DNA
  source 1..33: mol_type = other DNA
gtgcarggcaccgacgtttcatctacgacggtg

SEQ ID NO: 25 - DNA, 60 bp, synthetic construct
  misc_feature 1..45: note = DNA
  misc_feature 46: note = RNA
  misc_feature 47..60: note = DNA
  source 1..60: mol_type = other DNA
  misc_feature 33^34: note = three carbon spacer
cccctccctcctccttcctcctgagggccaaaaaaaactccctcaagtgcaggcaccgac

SEQ ID NO: 26 - DNA, 72 bp, synthetic construct
  source 1..72: mol_type = other DNA
  misc_feature 36^37: note = three carbon spacer
  modified_base 1: mod_base = OTHER; note = iso deoxycytosine
ctatatatatatatataatggactccaaaaaaaaccggtcccattgcaaaaaagagttccaaggaagaaggg

SEQ ID NO: 27 - DNA, 54 bp, synthetic construct
  misc_feature 1..43: note = DNA
  misc_feature 44: note = RNA
  misc_feature 45..54: note = DNA
  source 1..54: mol_type = other DNA
  misc_feature 31^32: note = three carbon spacer
  modified_base 1: mod_base = OTHER; note = iso deoxycytosine
ccctcctcctccttcctcctgagggccaaaaaaaaccccctcaatgacctgctg

SEQ ID NO: 28 - DNA, 54 bp, synthetic construct
  misc_feature 1..41: note = DNA
  misc_feature 42: note = RNA
  misc_feature 43..54: note = DNA
  source 1..54: mol_type = other DNA
  misc_feature 30^31: note = three carbon spacer
  modified_base 1: mod_base = OTHER; note = iso deoxycytosine
catttatatattatttaagggacaccaaaaaaacctgtccccctatgacctgct

SEQ ID NO: 29 - DNA, 62 bp, synthetic construct
  misc_feature 1..4: note = DNA
  misc_feature 5: note = RNA
  misc_feature 6..50: note = DNA
  misc_feature 51: note = RNA
  misc_feature 52..61: note = DNA
  source 1..62: mol_type = other DNA
  modified_base 10: mod_base = OTHER; note = iso deoxycytosine
  misc_feature 39^40: note = three carbon spacer
tcctcctccccctcctcctccttcctctgagggccaaaaaaaaccccctcaatgacctgctg

SEQ ID NO: 30 - DNA, 60 bp, synthetic construct
  misc_feature 1..4: note = DNA
  misc_feature 5: note = RNA
  misc_feature 6..51: note = DNA
  misc_feature 52: note = RNA
  misc_feature 53..60: note = DNA
  source 1..60: mol_type = other DNA
  modified_base 10: mod_base = OTHER; note = iso deoxycytosine
  misc_feature 39^40: note = three carbon spacer
tcctcctctctatatattatttaagggacaccaaaaaaacctgtccccctatgacctgct

SEQ ID NO: 31 - DNA, 999 bp, synthetic construct
  source 1..999: mol_type = other DNA
tttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagggaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctcaatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtctgagagctccctggctcccacacggttcagataatccctgctgcattttaccctcatcatgcaccactttagccaaattctgtct

SEQ ID NO: 32 - DNA, 36 bp, synthetic construct
  source 1..36: mol_type = other DNA
gagggaggatatggcctgctgctggagatgctggac

SEQ ID NO: 33 - DNA, 37 bp, synthetic construct
  source 1..37: mol_type = other DNA
ggagggaggatatggcctgctgctggagatgctggac

SEQ ID NO: 34 - DNA, 25 bp, synthetic construct
  misc_feature 1..13: note = DNA
  misc_feature 14: note = RNA
  misc_feature 15..25: note = DNA
  source 1..25: mol_type = other DNA
gagggaggatatggcctgctgctgg

SEQ ID NO: 35 - DNA, 75 bp, synthetic construct
  misc_feature 1..46: note = DNA
  misc_feature 47: note = RNA
  misc_feature 48..75: note = DNA
  source 1..75: mol_type = other DNA
  modified_base 1: mod_base = OTHER; note = iso deoxycytosine
  misc_feature 38^39: note = three carbon spacer
catttatatattatttaatgaacacaccaaaaaaaacctgtgttcactctataaacatatcctccctccttctct

SEQ ID NO: 36 - DNA, 15 bp, synthetic construct
  source 1..15: mol_type = other DNA
gatgcccctccacgg

SEQ ID NO: 37 - DNA, 167 bp, synthetic construct
  source 1..167: mol_type = other DNA
ttctgtgtcttcccacctacagtaacaaaggcatggagcatctgtacagcatgaagtgcaagaacgtggtgcccctctatggcctgctgctggagatgctggacgcccaccgcctacatgcgcccactagccgtggaggggcatccgtggaggagacggaccaaagc

SEQ ID NO: 38 - DNA, 23 bp, synthetic construct
  misc_feature 1..12: note = DNA
  misc_feature 13: note = RNA
  misc_feature 14..23: note = DNA
  source 1..23: mol_type = other DNA
gggaggcatctgcccagcaccct

SEQ ID NO: 39 - DNA, 22 bp, synthetic construct
  misc_feature 1..11: note = DNA
  misc_feature 12: note = RNA
  misc_feature 13..22: note = RNA
  source 1..22: mol_type = other DNA
ggaggcatctgcccagcaccct

SEQ ID NO: 40 - DNA, 27 bp, synthetic construct
  misc_feature 1..16: note = DNA
  misc_feature 17: note = RNA
  misc_feature 18..27: note = DNA
  source 1..27: mol_type = other DNA
  misc_feature 10^11: note = 3C spacer
gcgaagggagcatctgcccagcaccct

SEQ ID NO: 41 - DNA, 31 bp, synthetic construct
  misc_feature 1..11: note = DNA
  misc_feature 12: note = RNA
  misc_feature 13..31: note = DNA
  source 1..31: mol_type = other DNA
  misc_feature 16^17: note = three carbon spacer
ggaggcatctgcccagaccctgaagtctctg

SEQ ID NO: 42 - DNA, 73 bp, synthetic construct
  misc_feature 1..44: note = DNA
  misc_feature 45: note = RNA
  misc_feature 46..73: note = DNA
  source 1..73: mol_type = other DNA
  modified_base 1: mod_base = OTHER; note = iso deoxycytosine
  misc_feature 37^38: note = three carbon spacer
cccctataattcctatcttccaggctcaaaaaaaaccgcctggacctttaacacagatgcctcccttcgcctc

SEQ ID NO: 43 - DNA, 75 bp, synthetic construct; identical to SEQ ID NO: 35
  misc_feature 1..46: note = DNA
  misc_feature 47: note = RNA
  misc_feature 48..75: note = DNA
  source 1..75: mol_type = other DNA
  modified_base 1: mod_base = OTHER; note = iso deoxycytosine
  misc_feature 38^39: note = three carbon spacer

SEQ ID NO: 44 - DNA, 73 bp, synthetic construct; identical to SEQ ID NO: 42
  misc_feature 1..44: note = DNA
  misc_feature 45: note = RNA
  misc_feature 46..73: note = DNA
  source 1..73: mol_type = other DNA
  modified_base 1: mod_base = OTHER; note = iso deoxycytosine
  misc_feature 37^38: note = three carbon spacer

SEQ ID NO: 45 - DNA, 167 bp, synthetic construct
  source 1..167: mol_type = other DNA
ctctgggtctcctagacctcatcctctttgagcttctctctctcactctctctctgcgcattcaggagtgtacacatttctgcccagcaccctgaagtctctggaagagaaggaccatatccaccgagtcctggacaagatcacagacactttgatccacctgatgg

(19) United States
(12) Patent Application Publication
(10) Pub. No.: US 2023/0348957 A1
(43) Pub. Date: Nov. 2, 2023

(54) METHODS AND COMPOSITIONS FOR NUCLEIC ACID ANALYSIS

(71) Applicant: LUMINEX CORPORATION, Austin, TX (US)

(72) Inventor: Douglas WHITMAN, Austin, TX (US)

(73) Assignee: LUMINEX CORPORATION, Austin, TX (US)

(21) Appl. No.: 18/062,918

(22) Filed: Dec. 7, 2022

Related U.S. Application Data
(60) Provisional application No. 63/286,717, filed on Dec. 7, 2021.

(51) Int. Cl. C12Q 1/6818; C12Q 1/6851

(52) U.S. Cl. CPC C12Q 1/6818; C12Q 1/6851

(57) ABSTRACT

Methods and reagent for determining the presence and/or for quantifying the amount of a target nucleic acid sequences in a sample are provided. In some aspects, the methods comprise performing a melt analysis by detecting a signal from a hairpin probe that forms only in the presence of the target nucleic acid.

### REFERENCE TO RELATED APPLICATIONS

The present application claims the priority benefit of U.S. provisional application No. 63/286,717, filed Dec. 7, 2021, the entire contents of which are incorporated herein by reference.

### REFERENCE TO A SEQUENCE LISTING

This application contains a Sequence Listing XML, which has been submitted electronically and is hereby incorporated by reference in its entirety. Said Sequence Listing XML, created on Dec. 5, 2022, is named LUMNP0157US_ST26.xml and is 78,208 bytes in size.

### 1. FIELD

The present invention relates generally to the field of molecular biology. More particularly, it concerns methods and compositions for identifying nucleic acid targets in biological samples.

### 2. DESCRIPTION OF RELATED ART

Multiplex analysis of nucleic acids provides for the detection of multiple specific nucleic acid targets in a single reaction mixture. One way of detecting different nucleic acids in a reaction mixture is to use target-specific probes, wherein each different probe specific for each different target nucleic acid is labeled with a distinguishable fluorescent label. However, most commercially available fluorescent detection instruments are capable of distinguishing only 4-5 fluorophores, thus limiting the ability to detect more than 4-5 target nucleic acids in a reaction mixture.

One way to address this deficiency is to use probes having multidimensional signatures comprising a combination of T_{M} (the temperature at which 50% of a duplex nucleic acid is in double strand conformation and 50% of the duplex nucleic acid is in single strand conformation) and fluorescence of a specific wavelength. Melt analysis can be used to distinguish target-specific probes labeled with the same fluorescent label but having different T_{M}s from each other in a single reaction mixture. Probes used in melt analysis are designed to form duplexes in the presence of their cognate target nucleic acids that display different fluorescent signals when in a duplex conformation than in single-stranded conformation. For example, in aqueous solution, probes such as PLEIADES® probes are weakly fluorescent as a result of adopting a random coil structure that brings the fluor and quencher in close proximity, resulting in quenching of the fluorescent signal. Hybridization of PLEIADES® probes to their specific target nucleic acids results in an increase in fluorescence as a result of the linear conformation of the hybridized probe and associated increase in distance separating the fluor and quencher. Thermal denaturation of the probe from the target restores the weak fluorescence associated with the random coil configuration. MOLECULAR BEACON® probes exhibit minimal fluorescence in the absence of target nucleic acids at low temperatures as these probes adopt a stem loop configuration that brings the fluor and quencher into close proximity of each other. In the presence of target and at the annealing temperature, the loop region of the probe binds to the target nucleic acid, thus separating the flour and quencher, resulting in maximal fluorescence. Thermal denaturation of the probe and target nucleic acids permit the probe to adopt a random coil structure that emits weak fluorescence, similar to that for PLEIADES® probes. Such probe systems may be used in multiplex melt analysis, where probes specific for different target nucleic acids can be designed to have different T_{M}s representative of their cognate target nucleic acids. However, it can be challenging to design probe/target duplexes with predetermined distinguishable T_{M}s in the context of multiplex melt analysis, since target nucleic acid regions must be carefully selected to ensure the different probe/target duplexes have the requisite T_{M} values to be sufficiently distinguishable from each other. When probe T_{M} values are determined by natural sequences of target nucleic acids, it can be difficult to separate the probes from each other by T_{M} using the above traditional probe melt analysis techniques. Most PLEIADES® or MOLECULAR BEACON® probes will have a T_{M} between 55° C. and 70° C., with melt profiles that are overlapping and difficult to resolve when more than one target is present.

One way to address this challenge is to use probes that rely on a target-dependent cleavage event to activate the probe to form a monomeric hairpin structure having a predetermined T_{M} that is independent of the target nucleic acid sequence. WO2016/025452 teaches a method for detecting a target nucleic acid using a probe that includes a target nucleic acid-complementary region that includes a cleavage site. In the presence of the target nucleic acid, hybridization of the probe to the target nucleic acid results in cleavage at the cleavage site and subsequent intramolecular hybridization of internal complementary regions of the probe. Polymerase mediated extension at the cleaved 3′ end generates a monomeric double stranded stem of the hairpin probe. These hairpin probes can be designed to have predetermined T_{M}s by varying the nucleotide content and length of each stem such that each different Tm is representative of a specific target nucleic acid. The probes are thus suitable for use in multiplex melt analysis as probes for different targets can be designed to have distinguishable T_{M}s within a wide temperature range (55° C. to 90° C.) used for melt analysis. Since the target complementary region of these probes bind directly to the target, each different probe represents a different target nucleic acid sequence. This may present a challenge in probe design since a distinct probe must be designed for each different target nucleic acid to be detected in a reaction mixture. Further improvements to probes suitable for use in multiplex melt analysis are desired.

### 3. SUMMARY

In one embodiment, provided herein are methods for detecting a first target nucleic acid in a sample, the method comprising the steps of:
a) contacting the sample with reagents comprising:
   i) a first activation probe comprising from 5′ to 3′, a first region that is not complementary to the first target nucleic acid, a second region comprising a first cleavage region, wherein the second region is complementary to the first target nucleic acid, wherein the first cleavage region is capable of forming a first cleavage site when double stranded;
   ii) a first cleavable probe comprising from 5′ to 3′, a first region comprising a label, a second region, a loop region comprising a 3′ extension blocker at the 3′ end, a third region that is complementary to at least a portion of the second region, and a fourth region comprising a first cleavage region at the 5′ end, wherein at least a portion of the fourth region 3′ of the first cleavage region is complementary to the first region of the first activation probe, wherein the first cleavage region is capable of forming a first cleavage site when double stranded; and
   iii) one or more nucleases capable of cleaving the first cleavage site of the first activation probe and the first cleavage site of the first cleavable probe;
b) performing a reaction to form a first hairpin probe having a first T_{M} if any first target nucleic acid is present in the sample, wherein the reaction includes the steps of:
   i) specifically hybridizing the second region of the first activation probe to any first target nucleic acid, thereby forming a first cleavage site at the first cleavage region of the first activation probe;
   ii) cleaving the first cleavage site at the first cleavage region of the activation probe to release a first flap probe, comprising the first region of the first activation probe and at least one nucleotide of the second region of the first activation probe, from the hybridized first activation probe;
   iii) hybridizing the first flap probe to the fourth region of the first cleavable probe;
   iv) extending the hybridized first flap probe past the first cleavage region of the first cleavable probe using the first cleavable probe as a template, thereby forming a first cleavage site at the first cleavage region of the first cleavable probe;
   v) cleaving the first cleavage site at the first cleavage region of the first cleavable probe to form a first truncated probe; and
   vi) hybridizing the second and third regions of the first truncated probe and extending the hybridized third region to form a first hairpin probe having a first T_{M}; and
c) detecting the presence of the first target nucleic acid by detecting the presence of the first hairpin probe.

In some aspects, step b) further comprises performing an amplification reaction to amplify any first target nucleic acid present in the sample.

In some aspects, the label is a first member of a reporter-quencher pair and extension of the hybridized third region to form the first hairpin probe results in incorporation of the second member of the reporter-quencher pair at a location that permits interaction of the first and second members of the reporter-quencher pair. In some aspects, the first and second members of the reporter-quencher pair are each coupled to complementary non-natural bases respectively.

In some aspects, the non-natural bases are isoC and isoG.

In some aspects, the label comprises a reporter-quencher pair arranged such that the quencher quenches the reporter signal when the first region of the first cleavable probe is single stranded and extension of the hybridized third region separates the reporter and quencher to release the reporter from quenching.

In some aspects, the hairpin probe is detected by performing melt analysis. In some aspects, the hairpin probe is detected by detecting signals from the reporter at at least one temperature below the first T_{M} and at at least one temperature above the first T_{M} and detecting the presence of the target nucleic acid when a difference between the signal detected at the at least one temperature below the first T_{M} and the signal detected at the at least one temperature above the first T_{M} is detected.

In some aspects, the first activation probe is cleaved by an invader assay cleavage event. In some aspects, the first cleavage region of the first activation probe comprises at least one ribonucleotide and the first activation probe is cleaved by an endoribonuclease. In some aspects, the endoribonuclease is RNase HII. In some aspects, the first activation probe is cleaved by a polymerase having 5′ nuclease activity.

In some aspects, the fourth region of the first cleavable probe comprises one or more ribonucleotides and cleavage of the first cleavage region of the first cleavable probe is performed by RNase HII. In some aspects, cleavage of the first cleavable probe is performed by a restriction enzyme or a nicking enzyme.

In some aspects, the methods are multiplex methods further comprising detecting the presence of a second target nucleic acid wherein the method further comprises the steps of:
a) contacting the sample with reagents comprising:
   i) a second activation probe comprising from 5′ to 3′, a first region that is not complementary to the first or second target nucleic acids, a second region comprising a first cleavage region, wherein the second region is complementary to the second target nucleic acid, wherein the first cleavage region is capable of forming a first cleavage site when double stranded;
   ii) a second cleavable probe comprising from 5′ to 3′, a first region comprising a label, a second region, a loop region comprising a 3′ extension blocker at the 3′ end, a third region that is complementary to at least a portion of the second region, and a fourth region comprising a first cleavage region at the 5′ end, wherein at least a portion of the fourth region 3′ of the first cleavage region is complementary to the first region of the second activation probe, wherein the first cleavage region is capable of forming a first cleavage site when double stranded; and
   iii) one or more nucleases capable of cleaving the first cleavage site of the second activation probe and the first cleavage site of the second cleavable probe;
b) performing a reaction to form a second hairpin probe having a second T_{M} if any second target nucleic acid is present in the sample, wherein the reaction includes the steps of:
   i) specifically hybridizing the second region of the second activation probe to any second target nucleic acid, thereby forming a first cleavage site at the first cleavage region of the second activation probe;
   ii) cleaving the first cleavage site of the second activation probe to release a second flap probe, comprising the first region of the second activation probe and optionally between one and four nucleotides of the second region of the second activation probe, from the hybridized second activation probe;
   iii) hybridizing the second flap probe to the fourth region of the second cleavable probe;
   iv) extending the hybridized second flap probe past the first cleavage region of the second cleavable probe using the second cleavable probe as a template, thereby forming a first cleavage site at the first cleavage region of the second cleavable probe;
   v) cleaving the first cleavage site of the second cleavable probe to form a second truncated probe;
   vi) hybridizing the second and third regions of the second truncated probe and extending the hybridized third region to form a second hairpin probe having a second T_{M} that is different than the first T_{M} of the first hairpin probe; and
c) detecting the presence of the second target nucleic acid by detecting the presence of the second hairpin probe.

In some aspects, step b) further comprises performing an amplification reaction to amplify any second target nucleic acid present in the sample.

In some aspects, the labels on the first and second cleavable probes comprise the same reporter and the first T_{M} and second T_{M} are at least 5 degrees Celsius different, and wherein the first and second hairpins are detected by performing the steps of:
a) detecting signal from the reporter at a first temperature that is below the first T_{M} and at a second temperature that is above the first T_{M};
b) detecting signal from the reporter at a third temperature that is below the second T_{M} and a fourth temperature that is above the second T_{M}, wherein the third temperature is equal to or greater than the second temperature; and
c) detecting the presence of the first target nucleic acid when there is a difference between signals detected at the first and second temperatures, and detecting the presence of the second target nucleic acid when there is a difference between signals detected at the third and fourth temperatures.

In some aspects, the methods further comprise detecting the presence of a second target nucleic acid in the sample wherein the method further comprises the steps of:
a) contacting the sample with reagents comprising:
   i) a second activation probe comprising from 5′ to 3′, a first region that is not complementary to the first or second target nucleic acids and has the same sequence as the first region of the first activation probe, and a second region comprising a first cleavage region, wherein the second region is complementary to the second target nucleic acid, wherein the first cleavage region is capable of forming a first cleavage site when double stranded; and
   ii) one or more nucleases capable of cleaving the first cleavage site of the second activation probe;
b) performing a reaction to form the first hairpin probe having the first T_{M} if any second target nucleic acid is present in the sample, wherein the amplification reaction includes the steps of:
   i) specifically hybridizing the second region of the second activation probe to the second target nucleic acid, thereby forming a first cleavage site at the first cleavage region of the second activation probe;
   ii) cleaving the first cleavage site of the second activation probe to release a second flap probe, comprising the first region of the second activation probe and at least one nucleotide of the second region of the second activation probe, from the hybridized second activation probe;
   iii) hybridizing the second flap probe to the fourth region of the first cleavable probe;
   iv) extending the hybridized second flap probe past the first cleavage site of the first cleavable probe using the first cleavable probe as a template, thereby forming a first cleavage site at the first cleavage region of the first cleavable probe;
   v) cleaving the first cleavage site of the first cleavable probe to form the first truncated probe;
   vi) hybridizing the second and third regions of the first truncated probe and extending the hybridized third region to form the first hairpin probe having the first T_{M}; and
c) detecting the presence of the first or second target nucleic acid by detecting the presence of the first hairpin probe.

In some aspects, step b) further comprises performing an amplification reaction to amplify any second target nucleic acid present in the sample.

In some aspects, the methods further comprise detecting the presence of the first target nucleic acid or a second distinct target nucleic acid in the sample by detecting the presence of the first hairpin probe, wherein the reagents contacting the sample include a second activation probe having a 5′ first region that is not complementary to the first or second target nucleic acid and is the same as the 5′ first region of the first activation probe, and a second region comprising a first cleavage site, wherein the second region of the second activation probe is complementary to the second target nucleic acid.

In one embodiment, provided herein are methods for detecting a first target nucleic acid in a sample, the method comprising the steps of:
a) contacting the sample with reagents comprising:
   i) a first activation probe comprising from 5′ to 3′, a first region that is not complementary to the first target nucleic acid, a second region comprising a first cleavage region, wherein the second region is complementary to the first target nucleic acid, wherein the first cleavage region is capable of forming a first cleavage site when double stranded;
   ii) a first cleavable probe comprising from 5′ to 3′, a first region comprising a label, a second region, a loop region comprising a 3′ extension blocker at the 3′ end, a third region that is complementary to at least a portion of the second region, and a fourth region comprising a first cleavage region at the 5′ end, wherein the fourth region 3′ of the cleavage site is not complementary to the first region of the first activation probe, wherein the first cleavage region is capable of forming a first cleavage site when double stranded;
   iii) a conversion probe comprising from 5′ to 3′, a first region that has the same sequence as at least a portion of the fourth region of the first cleavable probe and a second region that is complementary to the released first region of the first activation probe; and
   iv) one or more nucleases capable of cleaving the first cleavage site of the first activation probe and the first cleavage site of the first cleavable probe;
b) performing a reaction to form a first hairpin probe having a first T_{M} if any first target nucleic acid is present in the sample, wherein the reaction includes the steps of:
   i) specifically hybridizing the second region of the first activation probe to any first target nucleic acid, thereby forming a first cleavage site at the first cleavage region of the first activation probe;
   ii) cleaving the first cleavage site at the first cleavage region of the activation probe to release a first flap probe, comprising the first region of the first activation probe and at least one nucleotide of the second region of the first activation probe, from the hybridized first activation probe;
   iii) hybridizing the released first flap probe to the conversion probe;
   iv) extending the hybridized first flap probe using the conversion probe as a template to form a modified first flap probe having a 3′ end that is complementary to the fourth region of the first cleavable probe;
   v) denaturing the modified first flap probe from the conversion probe for subsequent hybridization to the fourth region of the first cleavable probe
   vi) hybridizing the modified first flap probe to the fourth region of the first cleavable probe;
   vii) extending the hybridized modified first flap probe past the first cleavage region of the first cleavable probe using the first cleavable probe as a template, thereby forming a first cleavage site at the first cleavage region of the first cleavable probe;
   viii) cleaving the first cleavage site at the first cleavage region of the first cleavable probe to form a first truncated probe;
   ix) hybridizing the second and third regions of the first truncated probe and extending the hybridized third region to form a first hairpin probe having a first T_{M}; and
c) detecting the presence of the first target nucleic acid by detecting the presence of the first hairpin probe.

In some aspects, step b) further comprises performing an amplification reaction to amplify any first target nucleic acid present in the sample.

In some aspects, the first region of the conversion probe comprises one or more ribonucleotides. In some aspects, the methods further comprise, following step b)iv), cleaving the modified released first region of the first activation hybridized to the conversion probe.

In some aspects, the methods further comprise detecting the presence of a second target nucleic acid in the sample wherein the method further comprises the steps of:
a) contacting the sample with reagents comprising:
   i) a second activation probe comprising from 5′ to 3′, a first region that is not complementary to the first or second target nucleic acids and has the same sequence as the first region of the first activation probe, and a second region comprising a first cleavage region, wherein the second region is complementary to the second target nucleic acid, wherein the first cleavage region is capable of forming a first cleavage site when double stranded; and
   ii) one or more nucleases capable of cleaving the first cleavage site of the second activation probe;
b) performing a reaction to form the first hairpin probe having the first T_{M} if any second target nucleic acid is present in the sample, wherein the reaction includes the steps of:
   i) specifically hybridizing the second region of the second activation probe to the second target nucleic acid, thereby forming a first cleavage site at the first cleavage region of the second activation probe;
   ii) cleaving the first cleavage site of the second activation probe to release a second flap probe, comprising the first region of the second activation probe and at least one nucleotide of the second region of the second activation probe, from the hybridized second activation probe;
   iii) hybridizing the second flap probe to the conversion probe;
   iv) extending the hybridized second flap probe using the conversion probe as a template to form a modified second flap probe having a 3′ end that is complementary to the fourth region of the first cleavable probe;
   v) denaturing the modified second flap probe from the conversion probe for subsequent hybridization to the fourth region of the first cleavable probe;
   vi) hybridizing the modified second flap probe to the fourth region of the first cleavable probe;
   vii) extending the hybridized second flap probe past the first cleavage site of the first cleavable probe using the first cleavable probe as a template, thereby forming a first cleavage site at the first cleavage region of the first cleavable probe;
   viii) cleaving the first cleavage site of the first cleavable probe to form the first truncated probe;
   ix) hybridizing the second and third regions of the first truncated probe and extending the hybridized third region to form the first hairpin probe having the first T_{M}; and
c) detecting the presence of the first or second target nucleic acid by detecting the presence of the first hairpin probe.

In some aspects, step b) further comprises performing an amplification reaction to amplify any second target nucleic acid present in the sample.

In some aspects, the methods comprise detecting the presence of the first target nucleic acid or a second distinct target nucleic acid in the sample by detecting the presence of the first hairpin probe, wherein the reagents contacting the sample include a second activation probe having a 5′ first region that is not complementary to the first or second target nucleic acid and is the same as the 5′ first region of the first activation probe, and a second region comprising a first cleavage site, wherein the second region of the second activation probe is complementary to the second target nucleic acid.

In some aspects, the conversion probe further comprises between one and five nucleotides, positioned between the first region and the second region, that are complementary to the first one to five nucleotides of the target nucleic acid.

In some aspects, the conversion probe is further defined as a population of conversion probes, wherein each conversion probe comprises between one and five random nucleotides, positioned between the first region and the second region.

In one embodiment, provided herein are first cleavable probes for detecting a target nucleic acid, the probes comprising from 5′ to 3′, a first region comprising a label, a second region, a loop region comprising a 3′ extension blocker at the 3′ end, a third region that is complementary to at least a portion of the second region, and a fourth region comprising a first cleavage site at the 5′ end, wherein at least a portion of the fourth region 3′ of the cleavage site is complementary to a first region of a first activation probe, wherein the first activation probe comprises from 5′ to 3′, a first region that is not complementary to the target nucleic acid, a second region comprising a first cleavage site, wherein the second region is complementary to the target nucleic acid to be detected.

In some aspects, the label is a first member of a reporter-quencher pair. In some aspects, the label is coupled to a non-natural base. In some aspects, the non-natural base is isoC or isoG. In some aspects, the label comprises a reporter-quencher pair arranged such that the quencher quenches the reporter signal when the first region of the cleavable probe is single stranded.

In some aspects, the first cleavage site of the first cleavable probe comprises at least one ribonucleotide.

In one embodiment, provided herein are kits, or kits of parts, comprising the first cleavable probes of any one of the present embodiments.

In some aspects, the kits further comprises a first activation probe comprising from 5′ to 3′, a first region that is not complementary to the first target nucleic acid, a second region comprising a first cleavage site, wherein the second region is complementary to the first target nucleic acid. In some aspects, the first cleavage site of the first activation probe comprises at least one ribonucleotide.

In some aspects, the kits further comprise a first conversion probe comprising from 5′ to 3′, a first region that has the same sequence as at least a portion of the fourth region of the first cleavable probe and a second region that is complementary to the first region of the first activation probe.

In one embodiment, provided herein are compositions comprising the first cleavable probe of any one of the present embodiments and a first activation probe comprising from 5′ to 3′, a first region that is not complementary to the first target nucleic acid, a second region comprising a first cleavage site, wherein the second region is complementary to the first target nucleic acid. In some aspects, the first cleavage site of the first activation probe comprises at least one ribonucleotide.

In some aspects, the compositions further comprise a first conversion probe comprising from 5′ to 3′, a first region that has the same sequence as at least a portion of the fourth region of the first cleavable probe and a second region that is complementary to the first region of the first activation probe.

In one embodiment, provided herein are methods for detecting a first target nucleic acid in a sample, the method comprising the steps of:
a) contacting the sample with reagents comprising:
   i) a first cleavable probe comprising, from 5′ to 3′, a first region comprising a quencher positioned 5′ to a first cleavage region, a second region comprising a reporter labeled non-natural nucleotide, a third region comprising a 3′ extension blocker at the 3′ end, a fourth region that is complementary to at least a portion of the third region, a second cleavage region, and a fifth region, wherein at least a portion of the first cleavable probe is complementary to the first target nucleic acid, wherein the portion of the first cleavable probe that is complementary to the first target nucleic acid spans the 3′ end of the fourth region, the second cleavage region, and the 5′ end of the fifth region;
   ii) one or more nucleases capable of cleaving the first cleavage site of the first cleavable probe and the second cleavage site of the second cleavable probe;
b) performing a reaction to form a first hairpin probe having a first T_{M} if any first target nucleic acid is present in the sample, wherein the reaction includes the steps of:
   i) specifically hybridizing the first target nucleic acid to the first cleavable probe, thereby forming a cleavage site at the second cleavage region of the first cleavable probe;
   ii) cleaving the first cleavable probe at the cleavage site formed at the second cleavage region to form a first truncated probe;
   iii) hybridizing the third and fourth regions of the first truncated probe and extending the hybridized fourth region to form a hairpin probe having a first T_{M} and a quencher labeled non-natural nucleotide positioned opposite the reporter labeled non-natural nucleotide of the second region of the first cleavable probe and forming a cleavage site at the first cleavage region of the first cleavable probe;
   iv) cleaving the first cleavable probe at the cleavage site formed at the first cleavage region to release the first quencher from the first cleavable probe;
c) detecting the presence of the target nucleic acid by detecting the presence of the hairpin probe.

In some aspects, the reporter labeled non-natural nucleotide is one of iso-C or iso-G and the quencher labeled non-natural nucleotide is the other of iso-C or iso-G.

In some aspects, the first cleavable probe comprises an extension blocker at the 3′ end.

In some aspects, step b) further comprises performing an amplification reaction to amplify any first target nucleic acid present in the sample.

In some aspects, the hairpin probe is detected by performing melt analysis. In some aspects, the hairpin probe is detected by detecting signals from the reporter at at least one temperature below the first T_{M} and at at least one temperature above the first T_{M} and detecting the presence of the target nucleic acid when a difference between the signal detected at the at least one temperature below the first T_{M} and the signal detected at the at least one temperature above the first T_{M} is detected.

In some aspects, the first cleavage region of the first cleavable probe comprises one or more ribonucleotides and cleavage is performed by RNase HII. In some aspects, the first cleavage region of the first cleavable probe comprises one strand of an endonuclease cleavage site and cleavage is performed by a restriction enzyme or a nicking enzyme.

In some aspects, the second cleavage region of the first cleavable probe comprises one or more ribonucleotides and cleavage is performed by RNase HII. In some aspects, the second cleavage region of the first cleavable probe comprises one strand of an endonuclease cleavage site and cleavage is performed by a restriction enzyme or a nicking enzyme.

### 4. BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIGS. 1A-1D show exemplary mechanisms for cleavage of Activation Probes. FIG. 1A shows Invader Assay cleavage that occurs when an invasive oligonucleotide hybridizes upstream of an Activation probe that is hybridized to target nucleic acid to form a cleavage structure suitable for cleavage by a flap endonuclease; FIG. 1B shows endoribonuclease cleavage, where R represents a ribobase; FIG. 1C shows 5′ nuclease cleavage occurring as a result of extension of a primer hybridized upstream of the Activation Probe/target nucleic acid duplex. FIG. 1D shows endoribonuclease cleavage, where R represents a ribobase and unfilled triangles represent optional carbon-3 spacers. In all of FIGS. 1A-D, the solid triangle shown at the 3′ end of the Activation probes symbolizes an optional 3′ extension blocker and the arrow indicates the cleavage site.

FIGS. 2A-2E how an embodiment of the cleavable probes of the invention transition to hairpin probes as a result of cleavage of their cognate Activation probes in the presence of target nucleic acid. FIG. 2A shows an embodiment of an isobase labeled cleavable probe in the absence of cleaved activation probe, i.e. flap probe; FIG. 2B shows an embodiment of the cleavable probe after hybridization of the flap probe to the 3′ end thereof; FIG. 2C shows an embodiment of the cleavable probe after hybridization and extension of the flap probe to create a cleavage site within the cleavage region of the cleavable probe; FIG. 2D shows the resulting truncated probe of FIG. 2C after cleavage by an endoribonuclease, restriction enzyme, or nicking enzyme; FIG. 2E shows an embodiment of the extended hairpin probe resulting from extension of the free 3′ end of the truncated cleavage probe using the first region as a template. This extended hairpin probe having a predetermined Tm can then be used in melt analysis to detect presence of target nucleic acid. In all of FIGS. 2A through 2E, triangles represent 3′ extension blockers and “C” indicates the cleavage region of the cleavable probe. iC* and iG^{Q} represent isobases labeled with a fluor and quencher, respectively.

FIGS. 3A-3E show how an alternative embodiment of the cleavable probes of the invention transition to hairpin probes as a result of cleavage of their cognate Activation probes in the presence of target nucleic acid. FIG. 3A shows an embodiment of a random coil configuration cleavable probe in the absence of cleaved activation probe, i.e. flap probe; FIG. 3B shows an embodiment of the random coil cleavable probe after hybridization of the flap probe to the 3′ end thereof; FIG. 3C shows an embodiment of the random coil cleavable probe after hybridization and extension of the flap probe to create a cleavage site within the cleavage region of the cleavable probe; FIG. 3D shows the resulting truncated random coil cleavable probe of FIG. 3C after cleavage by an endoribonuclease, restriction enzyme, or nicking enzyme; FIG. 3E shows an embodiment of the extended random coil hairpin probe after extension of the free 3′ end of the truncated cleavage probe using the first region as a template. In all of FIGS. 3A through 3E, triangles represent 3′ extension blockers and “C” indicates the cleavage region of the cleavable probe. * and Q represent a flour and quencher, respectively.

FIG. 4 shows an embodiment of a conversion probe used in the method to convert flap probes having different 3′ termini to those having common 3′ termini. Conversion Probes are comprised of 2 regions—a first region that has the same sequence as the fourth region of its cognate cleavable probe, and a second region that is complementary to the 5′ non-hybridizing region of a flap probe as well as the variable nucleotides occurring at the 3′ end of a flap probe. Hybridization of the flap probe to the Conversion probe results in extension of the 3′ end of the flap probe to generate a region complementary to the fourth region of a cognate cleavable probe, permitting subsequent hybridization to the cleavable probe. Subsequent extension, cleavage and hairpin probe formation similar to that described for FIG. 2B-2E can then proceed. Symbols have the same meaning as described for FIG. 2. Conversion probes may include ribobases, such that after extension of the hybridized flap probe, cleavage sites, cleavable by RNase HII are formed. Cleavage at these sites results in fragmentation of the conversion probes at their 5′ end and subsequent dissociation of the duplex as a result of reduced duplex melting temperatures.

FIG. 5A-B shows an embodiment of the method using a dark cleavable probe to detect a target nucleic acid. Following hybridization of a flap probe to the dark cleavable probe and subsequent extension, cleavage and hairpin probe formation similar to that described for FIG. 2B-2E can proceed to generate a labeled hairpin probe with a predetermined TM for use in melt analysis. Symbols have the same meaning as described for FIG. 2.

FIGS. 6A-6B show the use of an activation probe and cleavable probe to create a target-specific signal. FIG. 6A shows the signal detected (upper) and melt curve (lower) for a reaction mixture having cleavable probe in the presence or absence of target and in the presence or absence of activation probe. FIG. 6B shows the signal detected (upper) and melt curve (lower) for a reaction mixture having cleavable probe and target but where the activation probe is either present, absent, or lacking a ribobase (NR).

FIGS. 7A-7B show the use of an activation probe and cleavable probe to create a target-specific signal in digital PCR. FIG. 7A shows the signal detected for a reaction mixture having activation probe, cleavable probe, and target nucleic acid. FIG. 7B shows the signal detected for a reaction mixture having cleavable probe and activation probe but where the target nucleic acid was absent.

FIGS. 8A-8F show the effects of using dark cleavable probes on signal:noise ratio in a background of varying concentrations of target specific to non-target specific probes. FIGS. 8A-8C show signal detected from a reaction mixture having a target nucleic acid and target specific dark cleavable probes in a background of non-target specific dark cleavable probes at ratios of 1:2, 1:3 and 1:4 respectively. FIGS. 8D-8F show signal detected from a reaction mixture having a target nucleic acid and target-specific cleavable probes in a background of non-target specific cleavable probes at ratios of 1:2, 1:3 and 1:4, respectively.

FIG. 9 shows the use of an activation probe and a cleavable probe to create a target-specific signal using a DNA polymerase containing exonuclease activity to cleave the activation probe. Detected signal (upper panel) and melt curve (lower panel) for a reaction mixture having a DNA polymerase with 5′-exonuclease activity and a cleavable probe in the presence or absence of target and in the presence or absence of an activation probe are shown.

FIGS. 10A-B show the use of an activation probe containing an internal C3 spacer and a cleavable probe to create a target-specific signal in real-time and digital PCR, respectively. The internal C3 spacer can be placed 5′ or 3′ of the ribonuclease binding region as needed to block DNA polymerase extension by a non-specific primer interaction without interfering in the activation of the cleavable probe. FIG. 10A shows the melt curve signal detection in reaction mixtures containing a cleavable probe in the presence or absence of target and in the presence or absence of an activation probe. FIG. 10B shows the signal detected for a reaction mixture containing a cleavable probe and an activation probe in the presence (left hand panel) or absence (right hand panel) of target.

### 5. DESCRIPTION

The present invention features compositions and methods for detecting one or more target nucleic acids in a reaction mixture. The compositions and methods may be used for multiplex melt analysis and utilize probes having predetermined T_{M}s that are not dependent on sequences associated with the particular target-nucleic sequences of interest. The compositions and methods described herein provide advantages such as ease of probe design, increased multiplex capability, and improved signal to noise ratios.

In some embodiments, methods and compositions for a detecting a target nucleic acid using a cleavable probe and an activation probe are provided. The cleavable probe is activated as a result of target-dependent cleavage of an activation probe and subsequent hybridization of a released fragment of the activation probe (i.e., a flap probe) to the cleavable probe or hybridization of a conversion probe-modified released fragment of the activation probe to the cleavable probe (i.e., a modified flap probe).

As used herein, the term “sample” generally refers to any material containing or suspected of containing a nucleic acid. A sample may include a bodily tissue or a bodily fluid including but not limited to blood (or a fraction of blood, such as plasma or serum), lymph, mucus, tears, urine, and saliva. A sample may comprise DNA (e.g., genomic DNA), RNA (e.g., mRNA), and/or cDNA, any of which may be amplified to provide an amplified nucleic acid. A sample may comprise material obtained from an environmental locus (e.g., a body of water, soil, and the like) or material obtained from a fomite (i.e., an inanimate object that serves to transfer pathogens from one host to another).

As used herein “nucleic acid” generally refers to a polymeric form of nucleotides of any length (e.g. at least 2, 3, 4, 5, 6, 10, 50, 100, 200, 500 or 1000 nucleotides), either deoxyribonucleotides or ribonucleotides or a combination thereof, and any modifications thereof. Modifications include, but are not limited to, those that provide other chemical groups that incorporate additional charge, polarizability, hydrogen bonding, electrostatic interaction, and fluxionality to the nucleic acid ligand bases or to the nucleic acid ligand as a whole. Accordingly, the nucleic acids described herein include not only the standard bases adenine (A), cytosine (C), guanine (G), thymine (T), and uracil (U) but also non-standard or non-natural nucleotides, analogs and derivatives thereof. Non-standard or non-natural nucleotides such as isoC or isoG, are described, for example, in U.S. Pat. Nos. 5,432,272, 5,965,364, 6,001,983, 6,037,120, and 6,140,496, all of which are incorporated herein by reference and include bases other than A, G, C, T, or U that can be incorporated into a growing nucleic acid strand by a polymerase and are capable of base-pairing with a complementary non-standard or non-natural nucleotide to form a base pair.

As used herein, “target,” “target sequence” or “target nucleic acid” refers to a nucleic acid sequence of interest. A “target,” “target sequence” or “target nucleic acid” may also be a surrogate nucleic acid sequence that is formed in the presence of a nucleic acid sequence of interest in a sample and is representative of the nucleic acid sequence of interest. The surrogate nucleic acid sequence may be an amplification product or a cleavage product that is generated upon specific binding to a nucleic acid sequence of interest.

An oligonucleotide is a nucleic acid that includes at least two nucleotides. An oligonucleotide may be designed to function as a “primer.” A “primer” is a short nucleic acid, usually a ssDNA oligonucleotide, which may be hybridized to a target nucleic acid by complementary base-pairing. The primer may then be extended along the target nucleic acid template strand by a polymerase enzyme, such as a DNA polymerase enzyme or an RNA polymerase enzyme. Primer pairs can be used for amplification (and identification) of a target nucleic acid sequence (e.g., by the polymerase chain reaction (PCR)). An oligonucleotide may be designed to function as a “probe.” A “probe” refers to an oligonucleotide or portions thereof, used to detect complementary target nucleic acid sequences. Probes or primers may include a detectable label. Probes may also be extended by a polymerase using a template nucleic acid. The template nucleic acid may be a partially self-complementary region of the probe or the template may be an independent partially complementary nucleic acid.

A primer or probe that is specific for a target nucleic acid will “hybridize” to the target nucleic acid under suitable conditions. As used herein, “hybridization” or “hybridizing” refers to the process by which an oligonucleotide single strand anneals with a complementary strand through base pairing under defined hybridization conditions. “Specific hybridization” is an indication that two nucleic acid sequences share a high degree of complementarity, and two nucleic acid strands that specifically hybridize are said to be “complementary.” Specific hybridization complexes form under permissive annealing conditions. Permissive conditions for annealing of nucleic acid sequences are routinely determinable by one of ordinary skill in the art and may occur, for example, at 65° C. in the presence of about 6×SSC. Annealing temperatures are typically selected to be about 5° C. to 20° C. lower than the thermal melting point (T_{M}) for the specific sequence at a defined ionic strength and pH. The T_{M} is the temperature (under defined ionic strength and pH) at which 50% of the target sequence hybridizes to a perfectly matched probe. Equations for calculating T_{M}, for example, nearest-neighbor parameters, and conditions for nucleic acid hybridization, are known in the art.

Melt analysis refers to a process by which hybridization between complementary strands of a labeled nucleic acid duplex is reversed and changes in signal are monitored as the strands dissociate. Starting at or below a temperature at which the duplex is stable, the temperature is increased above the duplex T_{M} to a temperature at which all duplex molecules become fully dissociated. The hybridization state of the duplex can be monitored through changes in signal such as those from a fluorophore and quencher pair as the duplex dissociates, and plotted as a function of relative fluorescence units (RFUs) versus temperature. In a plot of the negative derivative of RFU versus temperature (i.e. a melt curve), the T_{M} may be calculated from the peak of the curve. Ideally, signals are collected at relatively small temperature increments (for example every 0.5° C.), particularly within close range of the T_{M}, to generate sufficient data points to calculate an accurate T_{M}. In some cases, reassociation analysis may be performed rather than melt analysis. In reassociation analysis, the starting temperature is one at which the duplex is dissociated and signal is measured as the temperature is lowered to a temperature at which the duplex reassociates. While this application uses the term melt analysis to describe the monitoring of the hybridization state of the duplex as the temperature changes, a person skilled in the art understands that reassociation analysis may be substituted for melt analysis in the method of the invention. Melt analysis may not always require the determination of an accurate T_{M} of a duplex. In some situations, it may only be necessary to detect signal from a target duplex at a temperature at which all target duplexes are double stranded and a temperature at which all target duplexes are single stranded. A difference in signal at these two detection temperatures indicates that the duplex has transitioned from duplex to single strand conformation at the selected temperature interval.

As used herein, “amplification” or “amplifying” refers to the production of additional copies of a nucleic acid sequence or a target sequence. Amplification may be carried out using polymerase chain reaction (PCR) or other amplification technologies known in the art, such as isothermal amplification. The term “amplification reaction mixture” refers to an aqueous solution comprising the various reagents used to amplify a target nucleic acid. These may include enzymes (e.g., a thermostable polymerase), aqueous buffers, salts, amplification primers, target nucleic acid, nucleoside triphosphates, and optionally, at least one labeled probe and/or optionally, at least one agent for determining the melting temperature of an amplified target nucleic acid (e.g., a fluorescent intercalating agent that exhibits a change in fluorescence in the presence of double-stranded nucleic acid). In some embodiments, one or more primers or probes in the amplification mixture are labeled with a reporter that emits a detectable signal (e.g., a fluorophore). In some embodiments, the amplification mixture may include at least one nucleotide that is labeled with a quencher (e.g., Dabcyl). In some embodiments the probe may include both a fluorophore and a quencher.

As used herein, “digital polymerase chain reaction (dPCR)” refers to an amplification reaction where a sample is separated into a large number of partitions and the reaction is carried out in each partition individually. dPCR involves partitioning the sample such that individual nucleic acid molecules contained in the sample are localized in many separate regions, such as in individual wells in microwell plates or micropartitions, in the dispersed phase of an emulsion, or arrays of nucleic acid binding surfaces. Ideally, the majority of partitions will contain 0 or 1 copy of a target nucleic acid of interest, providing a negative or positive reaction, respectively. Unlike conventional PCR, dPCR is not dependent on the number of amplification cycles to determine the initial amount of the target nucleic acid in the sample. Accordingly, dPCR eliminates the reliance on exponential data to quantify target nucleic acids and provides absolute quantification. Bead emulsion PCR, which clonally amplifies nucleic acids on beads in an emulsion, is one example of a dPCR technique in which the reactions are portioned into droplets. See, e.g., U.S. Pat. Nos. 8,048,627 and 7,842,457, which are hereby incorporated by reference. When dPCR is performed in an emulsion, the emulsion should be heat stable to allow it to withstand thermal cycling conditions. In contrast, devices designed to form compartments or partitions in a static array on a planar surface may be used to perform dPCR. For example, WO2018094091, incorporated herein by reference, describes a device that directs fluids into partitions and subsequently isolates partitions from each other. Several other ways of forming static arrays or reaction chambers are have been described, for example as in U.S. Pat. Nos. 9,039,993, 9,643,178 PCT/US2003/041356, EP2906348 and Du et. al. “SlipChip” Lab on a Chip 9 (16):2286.

As used herein, a “3′ extension blocker” is a moiety associated with the 3′ terminal nucleotide of an oligonucleotide that prevents polymerase-mediated extension of the 3′ end. A 3′ extension blocker may be, without limitation, any of the following modified: 3′ddC, 3′ Inverted dT, 3′ C3 spacer, 3′ Amino, and 3′ phosphorylation.

As used herein, “reporters” or “labels” are chemical or biochemical moieties useful for labeling an oligonucleotide or nucleic acid. “Reporters” or “labels” include fluorescent agents, chemiluminescent agents, chromogenic agents, quenching agents, radionuclides, enzymes, substrates, cofactors, scintillation agents, inhibitors, magnetic particles, and other moieties known in the art. “Labels” or “reporters” are capable of generating a measurable signal and may be covalently or noncovalently coupled to an oligonucleotide or nucleotide using methods known in the art.

As used herein, a “fluorescent dye” or a “fluorophore” is a chemical group that can be excited by light to emit fluorescence at a given wavelength or range of wavelengths. Dyes that may be used in the disclosed methods include, but are not limited to, fluorophores such as ALEXA FLUOR™ dyes, Fluorescein, HEX™ or AQUAPHLUOR® and others known to those skilled in the art.

The oligonucleotides and nucleotides of the disclosed methods may be labeled with a quencher, suitable for quenching fluorescence of a fluorophore. Quenching may include dynamic quenching (e.g., by FRET), static quenching, or both.

A. Activation Probes

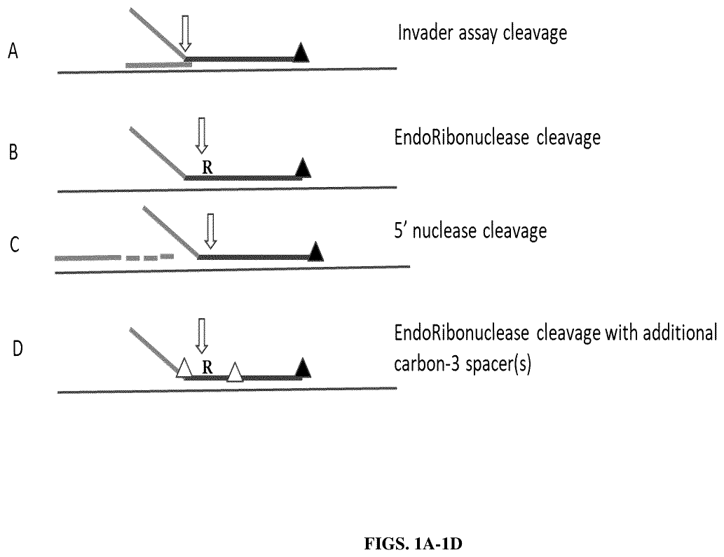

The activation probe used in the method has a 5′ first region that is not complementary to the target sequence but is complementary to a portion of a cleavable probe or a conversion probe, and a 3′ second region that is complementary to at least a portion of the target nucleic acid sequence and includes a cleavage region (FIG. 1). Optionally, the activation probe may include a 3′ extension blocker that prevents polymerase extension of the activation probe from the 3′ terminal nucleotide of the second region. Under specific hybridization conditions and in the presence of target nucleic acid, hybridization of the second region of the activation probe to the target nucleic acid occurs, thereby forming a cleavage site. In the presence of an appropriate nuclease, target binding-dependent cleavage occurs at the cleavage site to release a flap probe, which comprises the 5′ first region and nucleotide(s) from the 5′ end of the 3′ second region upstream of the cleavage site. The number of nucleotides from the 5′ end of the 3′ second region included in the flap probe depends on the type of cleavage mechanism utilized. Activation probes that utilize exonuclease cleavage are typically cleaved between 1 and 3 bases downstream of the 5′ end of the 3′ second region. Flap probes resulting from cleavage by a ribonuclease include all nucleotides upstream of the ribobase in the target binding region of the activation probe. Activation probes may include carbon 3 (C3) spacer modifications upstream of the ribobase, preferably at the junction between the flap region and the target binding region. This C3 modification can improve specificity by preventing non-specific cleavage and extension when an uncleaved flap probe hybridizes to a cleavable probe. It does this by disrupting the structure near the cleavage site, creating an anchor-footer structure making it less stable and less susceptible to non-specific hybridization. Activation probes that contain a ribobase may also contain a C3 spacer or other extension blocker between the ribobase and 3′ terminal extension blocker of the activation probe. This can help prevent non-specific extension of primers that hybridize non-specifically to the region between the ribobase and the 3′ terminal nucleotide of the activation probe.

Target binding-dependent cleavage at the cleavage site of the activation probe may be accomplished in a variety of ways known in the art (FIG. 1). For example, cleavage may occur as a result of an invader cleavage assay, as described in U.S. Pat. Nos. 7,691,573, 5,541,311 or Arruda et al., Expert Rev. Mol. Diagn., 2:487-496, 2002, or the similar QuARTS assay as described in U.S. Pat. No. 8,361,720 or Zou et al., Clin. Chem., 58:375-383, 2012. The invader cleavage assay (FIG. 1A) uses an invasive oligonucleotide (which may also be a primer) that hybridizes to the target nucleic acid upstream of an activation probe having a 5′ non-target hybridizing region and a 3′ target complementary region, such that the 3′ nucleotide of the invasive oligonucleotide overlaps the 5′ nucleotide of the 3′ target-complementary region of the activation probe by one nucleotide, creating a cleavage structure. This cleavage structure is recognized by a structure-specific flap endonuclease (FEN) that cleaves the activation probe after the 5′ nucleotide of the 3′ target complementary region of the activation probe, thus releasing the flap probe from the complex.

Other methods known in the art to cleave the 5′ non-target hybridizing region of an activation probe from the 3′ target hybridizing region of the activation probe include enzymatic cleavage of RNA:DNA hybrids (FIG. 1B). If the activation probe cleavage region includes a ribonucleotide base within the target hybridizing region, cleavage by an endonuclease, such as RNase HII, at the RNA:DNA hybrid formed when the target nucleic acid binds to the second region of the activation probe, can occur. Alternatively, cleavage and release of the flap probe may occur as a result of the 5′ exonuclease activity of some DNA polymerases during extension of an upstream primer (FIG. 1C). Both RNase HII-mediated cleavage and polymerase-mediated cleavage events cleave within the hybridized duplex formed by hybridization of the target-specific region of the activation probe to the target nucleic acid, resulting in cleavage occurring within the target hybridizing portion of the activation probe. The nucleotide sequence of the 3′ ends of the released flap in these embodiments are thus target-specific and may be variable.

As mentioned above, depending on the type of cleavage event and nuclease, the released flap probe may include between one and four target-specific nucleotides at its 3′ end. The number of target-specific nucleotides at the 3′ end of the released flap probe will depend on the method of cleavage. For the invader assay cleavage, it is expected that only one target-specific nucleotide will remain after cleavage. For endoribonuclease cleavage, it is expected that at least three target specific nucleotides could remain after cleavage, but the number can vary depending on where the ribonucleotide is located in the probe. For 5′ nuclease cleavage, the number of target specific nucleotides remaining can vary from one to four nucleotides.

B. Cleavable Probes

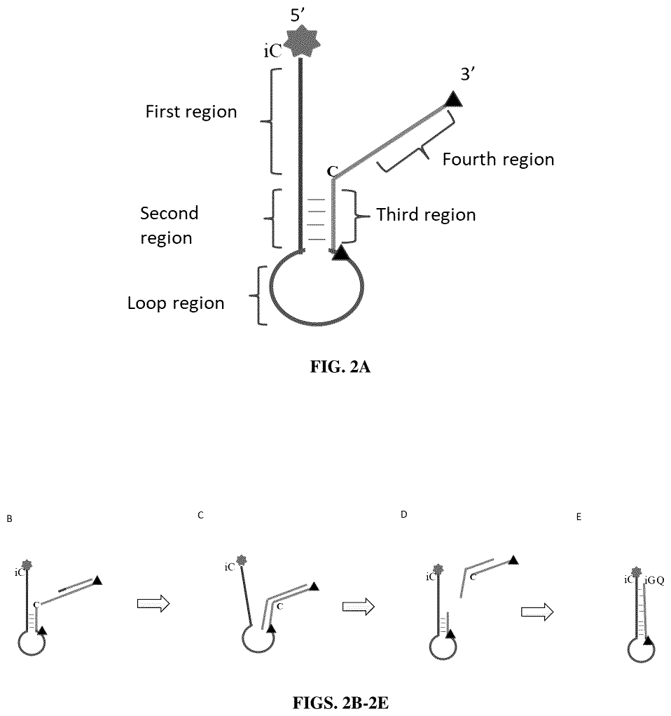

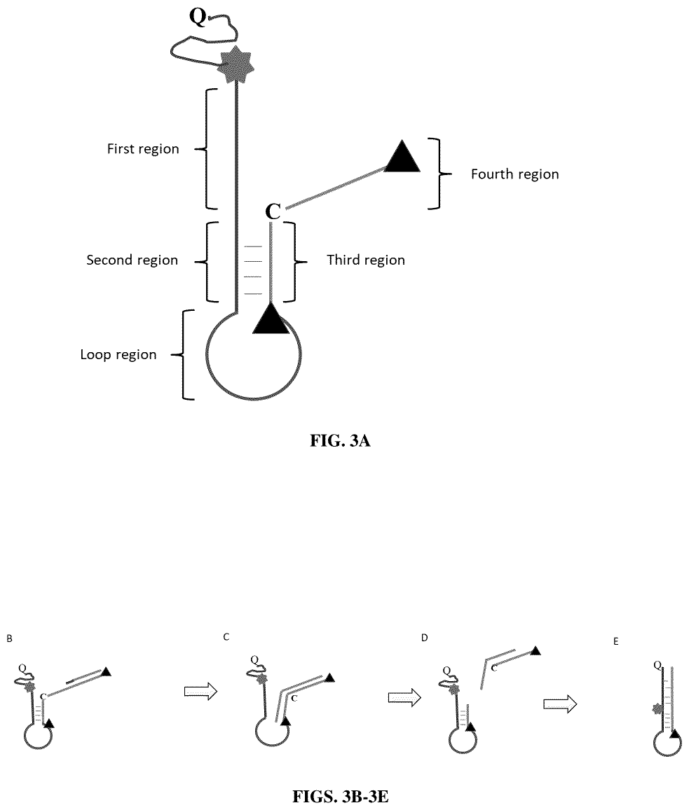

Cleavable probes for use in the methods have, from 5′ to 3′, a first region that includes a label, a second region, a loop region that has an extension blocker at its 3′ end, a third region that is capable of hybridizing with the second region, a cleavage region, and a fourth region (FIG. 2A). At least a portion of the fourth region is complementary to the released flap probe of the activation probe. Alternatively, at least a portion of the fourth region may be complementary to a modified flap probe, discussed further below. Optionally the label in the first region is at the 5′ end of the first region. Optionally the cleavable probe may include a 3′ extension blocker at the 3′ end.

Following release of a cleaved flap probe from the activation probe through target binding-dependent cleavage of the activation probe, the flap probe may hybridize with the fourth region of the cleavable probe, where it can be extended by polymerase, using the cleavable probe as a template, towards the 3′ extension blocker at the 3′ end of the loop region. This creates a cleavage site within the cleavage region between the fourth region and the third region (FIGS. 2B-2C). Cleavage at the cleavage site forms a truncated probe having a 3′ end that is suitable for extension (FIG. 2D). Hybridization of the second region of the truncated probe to the third region of the truncated probe allows for extension from the 3′ end to form a hairpin probe with a double stranded stem (FIG. 2E). The nucleotide sequence and length of the stem can be modulated to achieve a desired, predetermined T_{M} for use in melt analysis.

Cleavage of the cleavable probe may be achieved in a number of ways. For example, the cleavable probe may comprise one or more ribonucleotide(s) at the 5′ end of the fourth region. In this example, the released flap probe hybridizes to the fourth region of the cleavable probe at a position downstream of the ribonucleotide(s) and is extended by polymerase towards the 3′ extension blocker at the 3′ end of the loop region of the cleavable probe to create an RNA:DNA hybrid cleavage site for cleavage by RNase HII, as shown in FIG. 2C.

As another example, the cleavable probe may comprise one strand of a restriction enzyme or nicking enzyme recognition sequence overlapping the junction between the third region and the fourth region (FIG. 2D). Hybridization of the released flap probe at a position downstream of the restriction enzyme or nicking enzyme recognition sequence and subsequent extension of the 3′ end of the hybridized flap probe results in the formation of a cleavage site that may be recognized by the restriction enzyme or nicking enzyme for cleavage at the double stranded recognition sequence. The nicking enzyme endonuclease or restriction endonuclease is preferably thermal stable and may be engineered or naturally occurring. Double stranded cleavage, typical of restriction enzyme cleavage, or single strand cleavage of the cleavable probe may be utilized. A single stranded break may be preferable as it would allow the extended flap probe to hybridize to and cause cleavage of additional cleavable probes. Restriction enzymes may provide for single strand cleavage on cleavage sites comprising modified backbone linkages. For example, phosphorothioate (PTO), boranophosphate, methylphosphate, or peptide internucleotide linkages may be used to induce single stranded cleavage by some restriction enzymes. For example, FolI (NEB), BbsI (NEB), and BbsI (NEB) can be used with PTO. Naturally occurring nicking endonuclease cloned from Bacillus Sereothermophilus Nt.BstNBI can be used. PTO linkages can be produced by alpha thiol modified deoxynucleotides. Typically, the S isomer is employed which is incorporated into the nucleotide strand and confers nuclease resistance more effectively.

Examples of nicking endonucleases that may be used to cleave a double stranded cleavage site include, without limitation, Nb.BbvCI, Nb.BsmK, Nb.BsrDI, Nb.BssSI, Nb.BtsI, Nt.AIwI, Nt.BbvCI, Nt,BsmAI, Nt.BspQI, Nt.BstNBI, and Nt.CviPII.

Suitable labeling schemes can be incorporated into the cleavable probes to enable performing a melt analysis of the hairpin probes. For example, the label associated with the 5′ end of the first region may be a fluorescent reporter-quencher pair that adopts a random coil configuration in the absence of target nucleic acid (FIG. 3). In this single stranded conformation, the quencher is in close proximity to the reporter and thus quenches reporter signal. In the presence of target nucleic acid, extension of the hybridized third region to form a hairpin probe causes the random coil to adopt a linear double stranded conformation, thus separating the quencher and reporter to release the reporter from quenching (FIG. 3E). In another example, the label is a first member of a reporter-quencher pair and extension of the hybridized third region of the cleavable probe to form a hairpin probe results in incorporation of the second member of the reporter-quencher pair at a location that permits interaction of the first and second members (see FIGS. 2A-2E). A reporter may be attached to a non-natural nucleotide, for example iso-C or iso-G. If the extension reaction is performed in the presence of a quencher-labeled complementary non-natural nucleotide capable of base-pairing with the reporter-coupled non-natural nucleotide, incorporation of the complementary non-natural nucleotide results in quenching of the reporter signal. Either of these labeling schemes provides a hairpin probe suitable for use in melt analysis. Other labeling schemes may be utilized provided they result in a probe that emits a distinguishable signal in the presence of target nucleic acid and the labeling scheme is capable of distinguishing whether the probe is in a single-stranded conformation or double-stranded monomer conformation.

The activation probes and cleavable probes may be included in a reaction mixture that includes reagents suitable for amplification to generate an amplicon. Typical reagents for inclusion in a PCR amplification include DNA polymerases, dNTPs, and primers suitable for amplifying the target nucleic acid of interest. The reaction mixture may also include the requisite nucleases required for cleavage at the first cleavage site of the target nucleic acid-hybridized activation probe and the first cleavage site of the cleavable probe following flap probe-hybridization and extension.

Detecting the presence of the target nucleic acid involves detecting the presence of the hairpin probe having a predetermined T_{M}. The hairpin probe may be detected by performing melt analysis after amplification. In some embodiments, melt analysis may comprise subjecting the amplification reaction comprising the probe to small, incremental temperature increases (typically 0.1-0.5° C. per minute) while fluorescence is monitored continuously. In some embodiments, at temperatures below the hairpin's T_{M}, the fluorescent label of the hairpin probe is quenched by the quencher of the hairpin probe, and fluorescence increases slowly until the temperature approaches and passes the hairpin's T_{M}, enabling calculation of the hairpin probe's T_{M}. In some embodiments, at temperatures below the hairpin's T_{M}, the fluorescent label of the hairpin probe is not quenched by the quencher of the hairpin probe, and fluorescence decreases slowly until the temperature approaches and passes the hairpin's T_{M}, enabling calculation of the hairpin probe's T_{M}. In some embodiments, melt analysis may comprise detecting signals from the label at at least one temperature below the predetermined T_{M} and at least one temperature above the predetermined T_{M} for each probe in the reaction, but without requiring calculation of the T_{M} of each hairpin probe. In the presence of its cognate target nucleic acid, each hairpin probe will exhibit distinguishable signals at each of these temperatures. If multiple probes in the reaction are labeled with the same reporter but have sufficiently different predetermined T_{M} values, detecting a signal at a temperature below and a temperature above the predetermined T_{M} for each probe in the reaction at a series of non-overlapping temperature intervals enables the identification of melt profiles of hairpin probes in the reaction. In this way, it is possible to identify which of a number of different target nucleic acids is present in a reaction mixture or sample. Melt analysis can thus be utilized to identify the particular hairpin probe responsible for a detected change in signal depending on the temperature interval at which the change is detected.

C. Universal Cleavable Probes

A universal cleavable probe may be used to detect multiple different target nucleic acid sequences. In some clinical applications of nucleic acid detection, more than one target nucleic acid sequence of interest, for example multiple SNPs, may indicate the same clinical outcome, for example resistance to a chemotherapy drug or antibiotic. In these situations, while it is important for clinicians to be able to distinguish wild type sequence from variant sequences, it may not always be important to distinguish the identity of each and every variant sequence. Using a single universal probe that is representative of multiple variant nucleotides in a target nucleic acid would be beneficial, especially in a multiplex scenario as it would reduce the number of distinct probes in the reaction mixture and would increase the signal to noise ratio in the reaction, especially in detection schemes that rely on quenching of a fluorescent reporter in the presence of target nucleic acids.

First and second activation probes that specifically hybridize to respective first and second target nucleic acids may comprise the same 5′ first region that is not complementary to either the first or second target nucleic acid but is complementary to the fourth region of a universal cleavable probe. The first and second activation probes comprise 3′ target-hybridizing regions, each specific for a respective first or second target nucleic acid. Optionally, the first and second activation probes may include a 3′ extension blocker that prevents polymerase extension of the activation probe from the 3′ terminal nucleotide of the second region. Under specific hybridization conditions and in the presence of the first and second target nucleic acids, hybridization of the second region of each of the first and second activation probes to their respective first or second target nucleic acids occurs. As a result, target binding-dependent cleavage occurs at the cleavage site to release essentially the same flap probe from each of the different activation probes. Each flap probe comprises the same 5′ first region and between one and four nucleotide(s) of the 3′ second region, which may be the same or different for each flap probe depending on the target sequence.

As discussed for the activation probes described in Section A, target binding-dependent cleavage of the activation probe(s) may occur in a variety of ways. In some embodiments, activation probes may be cleaved in an invader assay cleavage event. In other embodiments, activation probes may include a ribobase in the target-complementary region and cleavage is due to an endoribonuclease present in the reaction. In other embodiments, cleavage may occur as a result of extension of an upstream primer by a polymerase having 5′ nuclease activity.

As mentioned above, for the invader assay cleavage, it is expected that only one target-specific nucleotide will remain on the flap probe after cleavage of the activation probe. Thus, when cleavage occurs as a result of an invader assay cleavage event, or any other cleavage method that results in one target-specific nucleotide remaining on the flap probe, a population of universal cleavable probes may be used. This population of universal cleavable probes will have identical fourth regions complementary to the 5′ non-target hybridizing region of the activation probes and a degenerate nucleotide at the 5′ end of the identical fourth regions. All four nucleotides may be represented at this position across different activation probes such that each flap probe will be able to hybridize to and be extended using one of the members of the population of universal cleavable probes as a template, regardless of the identity of the target-specific nucleotide present at the 3′ end of the flap probe.

Alternatively, activation probes may be grouped such that those that recognize multiple different target nucleic acids, but result in a cleaved flap probe having a 3′ A/T base pair, all have the same 5′ non-target hybridizing region/flap sequence that is complementary to the same universal cleavable probe. Similarly, flap probes that recognize multiple different target nucleic acids, but result in a cleaved flap probe ending in a G/C base pair may be grouped together and have the same 5′ non-target hybridizing region complementary to the same universal probe. In this way different target nucleic acid sequences having similar clinical utility can be represented by signal originating from a single population of universal cleavable probes within a group of different populations of cleavable probes, wherein each population has a representative multidimensional signature comprised of a unique reporter wavelength and a unique melt profile.

For cleavage methods that result in more than one target-specific nucleotide remaining on the flap probe, the above concept can be extended such that two or more nucleotides immediately 5′ of the region that is complementary to the 5′ non-target hybridizing region of the activation probes may be a degenerate nucleotide. However, it may be desirable to modify the flap probe prior to hybridizing to the universal cleavable probe so that the need to include degeneracy in the cleavable probe is reduced or eliminated.

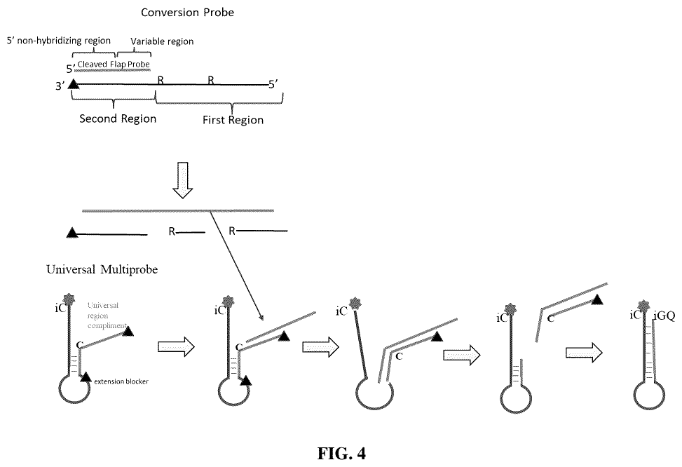

One way to accomplish the modification is to use a conversion probe, as shown in FIG. 4. A conversion probe comprises from 5′ to 3,′ a first region that has the same sequence as at least a portion of the fourth region of the cleavable probe representative of the target nucleic acid to be detected and a second region that is complementary to the released flap probe including the variable portion of the flap probe. Under suitable conditions, cleavage of the activation probe and subsequent hybridization of the released flap probe to the second region of a complementary conversion probe results in extension of the 3′ end of the released flap probe using the first region of the conversion probe as a template to introduce a sequence complementary to at least a portion of the fourth region of the cleavable probe into the released flap oligonucleotide at the 3′ end thereof. In some embodiments, the conversion probe may further include one or more ribonucleotides in the first region of the conversion probe such that extension of the hybridized released flap probe by polymerase results in generation of one or more RNA:DNA hybrid base pairs suitable for cleavage by RNase HII endoribonuclease. In the presence of RNase HII, cleavage at these sites results in destabilization of the hybridized duplex and dissociation of the newly extended flap probes from the cleaved conversion probe fragments. This modification thus prevents competition and favors hybridization of newly extended flap probes to their cognate cleavable probes for subsequent extension The extended flap oligonucleotide has a 3′ end that is complementary to a portion of the fourth region of the cleavable probe and can hybridize thereto. Conversion probes may be designed such that each conversion probe type is capable of converting a group of activation probes, wherein each member of the group is specific for different target nucleic acid sequences, to be complementary to the same universal probe type. Conversion probes of the same type may have the same 3′ regions complementary to the 5′ non-target hybridizing region of a group of activation probesand the same 5′ regions having the same sequence as the fourth region of a universal cleavable probe.

Use of conversion probes as described herein enables the conversion of distinct unlabeled flap probes that hybridize to different unique target genomic sequences to a common flap probe that hybridizes to a single cognate cleavage probe and thus generate a single melt signature. This increases the statistics in the reaction without sacrificing the signal to noise ratio of the reaction, since many distinct targets can be detected with the same labeled probe

D. Dark Cleavable Probes

Many probe systems used in nucleic acid detection utilize fluorescent reporters in combination with quenchers. In one of the hairpin probe systems described in WO2016/025452, the probe is labeled with a fluorescent reporter attached to a non-natural nucleotide. In the presence of target nucleic acid, the probe is modified to incorporate a complementary non-natural nucleotide labeled with a quencher capable of quenching the reporter signal. While this probe system enables the use of multiple different probes labeled with the same reporter in a single multiplex reaction by virtue of the fact that they can be distinguished by their unique melt profiles, the presence of the fluorescent reporter on unreacted probes results in high background fluorescence in the reaction. This presents particular challenges in the context of multiplex assays in which only one of a number of different target nucleic acids may be present in a sample and target nucleic acids are identified by melt analysis. In this situation, only one probe type will show a change in signal during melt analysis against a background of multiple fluorescent unreacted probe types. An alternative probe system may allow unreacted probes to remain quenched, while probes that have been modified due to the presence of their target nucleic acids in the sample are unquenched, and thus are capable of emitting distinguishable fluorescent signals to support performing melt analysis.

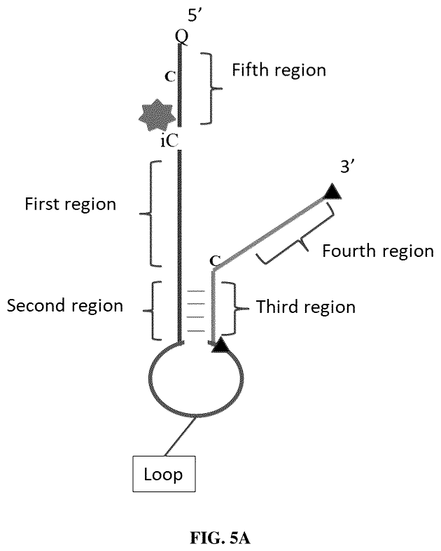

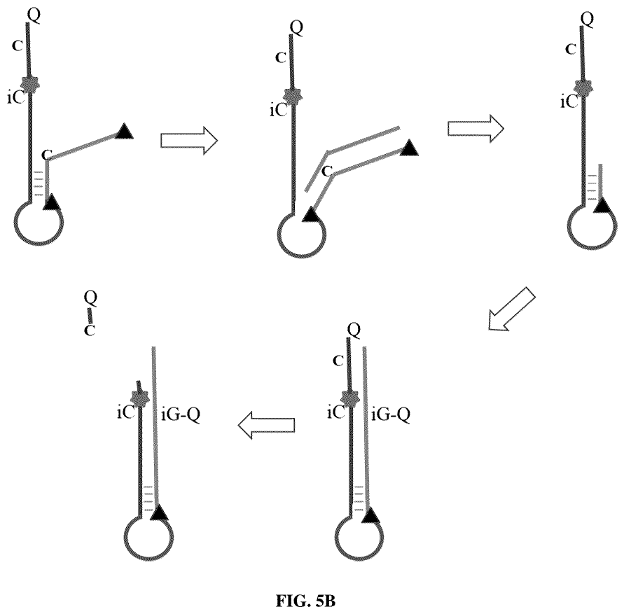

For example, compositions and methods for detecting nucleic acids are provided that make use of fluorescently labeled probes that remain quenched in the absence of their target nucleic acids, but are modified in the presence of their target nucleic acids to provide a unique melt profile that can be used to identify the presence of the target nucleic acid. The method utilizes a dark cleavable probe comprising the structure of the cleavable probes described above in section B, and further comprising a fifth region positioned at the 5′ end of the cleavable probe. The fifth region comprises a cleavage region and a quencher positioned 5′ of the cleavage region (FIG. 5A). The quencher of the fifth region is located at a position that allows it to quench signal from the label that is present in the first region. The cleavage region in the fifth region may comprise a ribobase or one strand of a restriction or nick enzyme site. The dark cleavable probe may optionally include a 3′ extension blocker at its 3′ end.

The dark cleavable probe may be included in a reaction mixture comprising the target nucleic acid, activation probes, and reagents for amplification of the target nucleic acid and cleavage of cleavage regions formed by hybridization of the activation probe to the target nucleic acid and extension of the flap probe on the dark cleavable probe. During amplification, conditions are provided for the flap probe to specifically hybridize to the fourth region of the dark cleavable probe and be extended to create a cleavage site (FIG. 5B). In the presence of a nuclease, such as RNase HII, cleavage occurs at the cleavage site to form a dark truncated probe with an extensible 3′ end. The third region of the dark truncated probe hybridizes to the second region and polymerase extension extends the 3′ end of the third region to form a hairpin probe having a non-natural nucleotide labeled with a second quencher positioned opposite the non-natural nucleotide in the first region, and positioned such that it can quench the label in the first region. Further Extension of the 3′ end creates a second cleavage site at the position of the cleavage region within the fifth region, resulting in cleavage by a ribonuclease and release of the quencher positioned 5′ to the cleavage region within the fifth region. The resultant hairpin probe is suitable for performing melt analysis and can be designed to have a predetermined T_{M} by varying the nucleotide content and length of each hairpin stem. The target nucleic acid is detected by detecting the presence of the hairpin probe, which may be detected by performing melt analysis.

In another embodiment, a dark cleavable probe can be designed such that the target nucleic acid hybridizes directly to the dark cleavable probe. In this case, the dark cleavable probe is designed so that the fourth region and at least 3 nucleotides at the 3′ end of the third region are complementary to the target nucleic acid. With this design, hybridization of the target nucleic acid to the dark cleavable probe will convert the cleavage region between the third and fourth regions of the dark cleavable probe into a cleavage site. Thus, when the target nucleic acid hybridizes to the dark cleavable probe, the cleavage site is cleaved to release the target nucleic acid hybridized to the complementary regions of the dark cleavable probe, allowing the truncated dark probe to self-hybridize and extend on itself from the newly formed 3′ end. Extension of the 3′ end creates a second cleavage site at the position of the cleavage region within the fifth region, resulting in cleavage by a ribonuclease and release of the quencher positioned 5′ to the cleavage region within the fifth region. The resultant hairpin probe is suitable for performing melt analysis and can be designed to have a predetermined T_{M} by varying the nucleotide content and length of each hairpin stem. The target nucleic acid is detected by detecting the presence of the hairpin probe, which may be detected by performing melt analysis.

### 6. EXAMPLES

Cleavable Probes with PCR

The following experiment was performed to demonstrate cleavage of a ribonucleotide-containing activation probe in the presence of a target nucleic acid during PCR, and subsequent hybridization to a cleavable probe, followed by extension to generate a cleavage site in the cleavable probe. This resulted in cleavage and extension of the cleavable probe to form a detectable hairpin probe. Melt analysis was performed after the PCR reaction.

| | | Y537S | WT | RMP-01 |
|---|---|---|---|---|
| Oligo | Sequence | T_{M} | T_{M} | T_{M} |
| Activation | CTTCTTCCTTGGAACTCTr [symbol] TGACCTGCTGC/3 | 62.5 | 52.7 | 57.2 |
| probe 1 | SpC/ (SEQ ID NO: 1) | | | |
| Cleavable | /FAM//isoC/TATATATATATATATATTGGAC | | | |
| Probe-01B | CCCAAAAAAAACC/iSpC3/GGTCCArUTGTaa | | | |
| | ataaaAGAGTTCCAAGGAAGAAGGG/ 3SpC3/ | | | |
| | (SEQ ID NO: 2) | | | |
| AP-NR- | CTTCTTCCTTGGAACTCT [symbol] TGACCTGCTGC/3S | | | |
| Ctrl | pc/ (SEQ ID NO: 3) | | | |

Primers and target sequences used for PCR amplification were as follows:

| Oligo/gblock | Sequence |
|---|---|
| JET401 | TGG GCG TCC AGC ATC TC (SEQ ID NO: 4) |
| LH1375 | CAA AGG CAT GGA GCA TCT GTA (SEQ ID NO: 5) |
| TW661 | TTCTGTGTCTTCCCACCTACAGTAACAAAGGCATGGAGCATCTGTACAGCA |
| | TGAAGTGCAAGAACGTGGIGCCCCTCTCTGACCTGCTGCTGGAGATGCTGG |
| | ACGCCCACCGCCTACATGCGCCCACTAGCCGTGGAGGGGCATCCGTGGAGG |
| | AGACGGACCAAAGC (SEQ ID NO: 6) |

The amplification mixture contained 10 mM Tris, 20 mM Bis-tris propane (BTP), 300 μg/mL BSA, 0.09 mM DTT, 2.5 mM MgCl₂, 50 mM KCl, 0.1 mM dNTPs, 1 μM Dabcyl-diGTP, 1× Titanium Taq Polymerase (Takara Biosciences), 16.2 mU/μL RNase HII (Takara Biosciences), 100 nM of JET401 oligonucleotide primer, 400 nM LH1375 oligonucleotide primer, and 50 nM of the Fam-labeled Cleavable Probe-01B. Activation Probe 1 oligos, when present, were at a concentration of 50 nM. The target nucleic acid (TW661) was spiked into the reaction at 10,000 copies or not at all in the NTC (no template control).

Thermal cycling was performed on an ABI 7500 Fast system (Thermo Fisher) at a final volume of 25 uL with the following conditions: 1 cycle of 2 min 20 s at 95° C.; 60 cycles of the following: 10 s at 95° C., 20 s at 58° C.

Measurements of the signal in the reaction were taken during PCR at the 58° C. stage of each cycle and after PCR at 0.5° C. increments starting at 60° C. and end at 95° C.

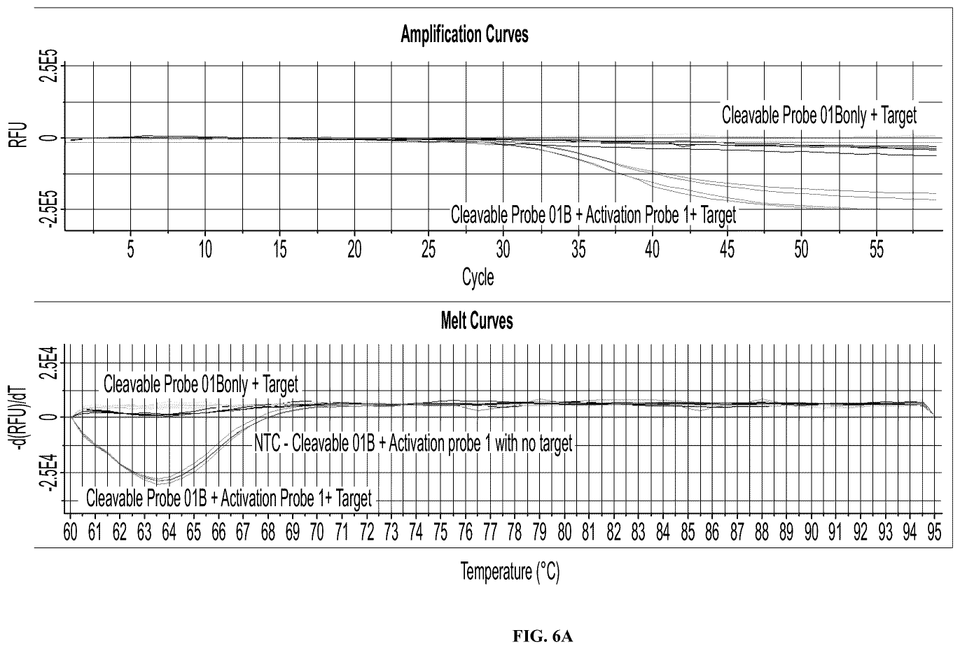

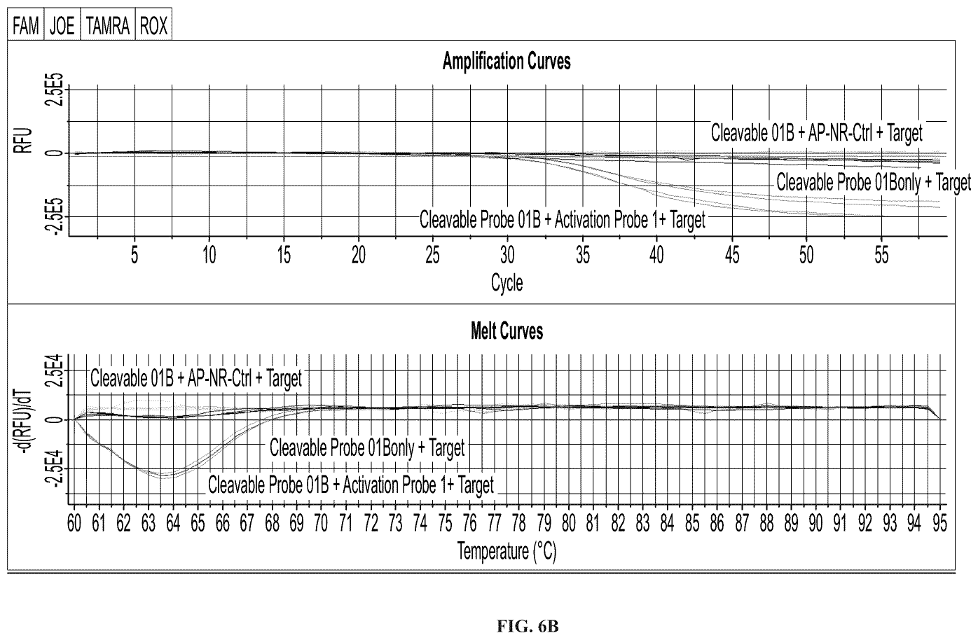

The reaction mixtures containing target, cleavable probe, and activation probe resulted in a decrease in FAM signal starting around cycle 30 during PCR and continuing to decrease throughout the PCR (FIG. 6A). Melt analysis performed after PCR showed a positive melt peak with a T_{M} (melting temperature) of 64° C. (FIG. 6A). The FAM signal at 60° C. was significantly less than the FAM signal at 68° C. for this condition.

Other conditions that did not have target present, or did not have activation probe present did not exhibit a change in signal during PCR, did not have a positive melt peak, and did not show a change in signal between 60° C. and 68° C. (FIG. 6A).

When Activation Probe 1 was replaced with AP-NR-Ctrl (the same as Activation Probe 1 except that the ribobase was replaced with DNA only) also did not exhibit a change in signal during PCR or after PCR during the melt analysis (FIG. 6B).

Cleavable Probes with dPCR

The following experiment was performed to demonstrate cleavage of a ribonucleotide-containing activation probe in the presence of a target nucleic acid during digital PCR (dPCR), and subsequent hybridization to a cleavable probe, followed by extension to generate a cleavage site in the cleavable probe. This resulted in cleavage and extension of the cleavable probe to form a detectable hairpin probe. Melt analysis was performed after the dPCR reaction.

| | | Y537S | WT | RMP-01 |
|---|---|---|---|---|
| Oligo | Sequence | T_{M} | T_{M} | T_{M} |
| Activation | CTTCTTCCTTGGAACTCTr [symbol] TGACCTGCTGC/3 | 62.5 | 52.7 | 57.2 |
| probe 1 | SpC/ (SEQ ID NO: 1) | | | |
| Cleavable | /FAM//isoC/TATATATATATATATATTGGAC | | | |
| Probe-01B | CCCAAAAAAAACC/iSpC3/GGTCCArUTGTaa | | | |
| | ataaaAGAGTTCCAAGGAAGAAGGG/3SpC3/ | | | |
| | (SEQ ID NO: 2) | | | |

Primers and target sequences used for PCR amplification were as follows:

| Oligo/gblock | Sequence |
|---|---|
| JET401 | TGG GCG TCC AGC ATC TC (SEQ ID NO: 4) |
| LH1375 | CAA AGG CAT GGA GCA TCT GTA (SEQ ID NO: 5) |
| TW661 | TTCTGTGTCTTCCCACCTACAGTAACAAAGGCATGGAGCATCTGTACAGCA |
| | TGAAGTGCAAGAACGTGGTGCCCCTCTCTGACCTGCTGCTGGAGATGCTGG |
| | ACGCCCACCGCCTACATGCGCCCACTAGCCGTGGAGGGGCATCCGTGGAGG |
| | AGACGGACCAAAGC (SEQ ID NO: 6) |

The amplification mixture contained 10 mM Tris, 20 mM Bis-tris propane (BTP), 300 μg/mL BSA, 0.09 mM DTT, 2.5 mM MgCl₂, 50 mM KCl, 0.1 mM dNTPs, 1 μM Dabcyl-diGTP, 2×Titanium Taq Polymerase (Takara Biosciences), 44.8 mU/μL RNase HII (Takara Biosciences), 100 nM of JET401 oligonucleotide primer, 400 nM LH1375 oligonucleotide primer, and 1200 nM of the Fam-labeled Cleavable Probe-01B. Activation Probe 1 oligos were at a concentration of 50 nM. The target nucleic acid (TW661) was spiked into the reaction at 1,000 copies or not at all in the NTC (no template control).

Digitization and thermal cycling were performed on an Applied Biosystems QuantStudio Absolute Q Digital PCR System. 9 μL of reaction mixture was transferred to the well of a MAP16 consumable and covered with 15 μL of Isolation Buffer (Applied Biosystems). Gaskets were appended to each well and the consumable was transferred to the dPCR system. The consumable was pressurized at 75 psi for 25 min and held at 50 psi to load and digitize the sample across approximately 20,000 partitions. Amplification was performed by pre-heating the consumable at 96° C. for 10 min., followed by 49 cycles of the following: 5 s at 93° C., 20 s at 60° C.; pressure was maintained at 50 psi throughout the PCR reaction. A 10 min isothermal hold at 55° C. was incorporated after PCR, but before imaging. After thermal cycling, Combinati analysis software was used to locate and identify partitions within the unit and to acquire images of partitions at 50° C. (T1), 72° C. (T2), 81° C. (T3), and 92° C. (T4) with optical filters set to preferentially obtain fluorescence values for the FAM-AP fluorophore. Ratios were calculated for fluorescence signals measured at successive temperatures and plotted on a 1D amplitude plot. The detectable hairpin probe formed from Cleavable Probe 01B after cleavage by Activation Probe 1 has a T_{M} of 63.5° C. and was thus expected to show maximum change in fluorescence (ratio >1) between T1 and T2.

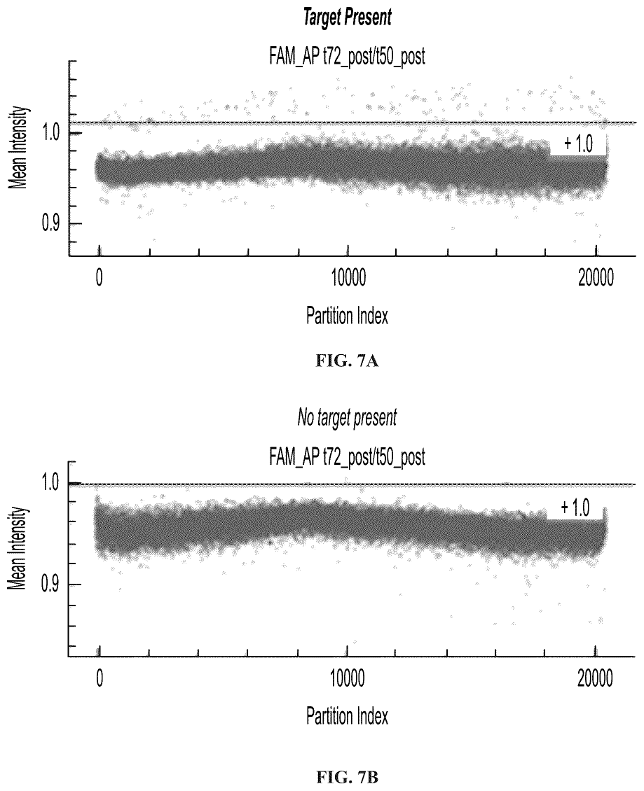

The reaction mixture containing 1200 nM Cleavable probe and 50 nM Activation probe resulted in a decrease in FAM signal between T1 (50° C.) and T2 (72° C.), resulting in a clear positive population in the 1D amplitude plot (FIG. 7A). In conditions where target was not present, there was no change in signal from T1 to T2, and no positive partitions were detected (FIG. 7B).

Universal Cleavable Probes with Invader Cleavage

In this example, two separate targets can be detected with a single Cleavable Probe. Forward and Reverse primers 1, amplify target 1 to create an amplicon, which Activation Probe 1-Inv and Invader 1 react with to cleave Activation Probe 1-Inv. The cleaved Activation Probe 1-Inv (i.e., Flap Probe 1-Inv) can now extend along the Cleavable Probe-Inv since it has an extensible 3′-OH group that is complimentary to the Cleavable Probe-Inv.

Forward and Reverse primers 2, can amplify target 2 to create an amplicon, which Activation Probe 2-Inv reacts with along with the Reverse primer 2 (also acting as an invader oligo) to cleave Activation Probe 2-Inv. The cleaved Activation Probe 2-Inv (i.e., Flap Probe 2-Inv) can now extend along the same Cleavable Probe-Inv since it has an extensible 3′-OH group that is complimentary to the Cleavable Probe-Inv, where it stops extending at the extension blocker. Because Activation Probe 2-Inv extended beyond the ribobase of Cleavable Probe-Inv, Cleavable Probe-Inv can now be cleaved at the ribobase via the RNaseHII enzyme. After the Cleavable Probe-Inv is cleaved, it can then denature and rehybridize to self during the denaturation and annealing phases of the next PCR cycle. When it rehybridizes to self, it can extend along itself, where it can attach a quencher across from the fluorophore via the dabcyl-isoGtp nucleoside quencher. This will cause a decrease in fluorescence during the reaction, and a specific melt signature during a melt analysis after PCR. A melt analysis is done by observing the signal in the reaction at more than 1 temperature. This demonstrates that a single cleavable probe can identify and quantify more than one target in a qPCR or digital PCR reaction.

Universal cleavable probe reactions can be performed using 400 nM of each of the primers and each activation probe, 100 nmol/L invasive oligo, 50 nM of the cleavable probe, 6.675 ng/μL cleavase 2.0 (Hologic), 1 U hotstart GoTaq polymerase (Promega), 11.2 mU RNaseH2, 20 mmol/L BTP pH 9.1, 10 mmol/L Tris pH 8.3, 50 mM KCl, 5 mmol/L MgCl₂, 0.3 mg/nL non-acetylated BSA, 1 μM Dabcyl-diGTP, 0.09 mmol/L DTT, and 250 μmol/L of each dNTP.

Cycling conditions can consist of 95° C. for 3 min; 10 cycles at 95° C. for 20 s, 67° C. for 30 s, and 70° C. for 30 s; 37 cycles at 95° C. for 20 s, 53° C. for 1 min, and 70° C. for 30 s; and 40° C. hold for 30 s.

Primers, probes, and target sequences used for are as follows:

| Molecule | Sequence |
|---|---|
| Activation | CCACTCTCTCTCTGATAGTCAGAGGTGAC/3SpC3/ (SEQ ID NO: 7) |
| probe 1-Inv | |
| Flap Probe 1- | CCACTCTCTCTCTGAT (SEQ ID NO: 8) |
| Inv | |
| Fwd Primer 1 | GGCTCTCATGGAATGGCTAAAGAC (SEQ ID NO: 9) (67C Tm) |
| Rev Primer 1 | ACGCTGCAGTCCTCGCTCA (SEQ ID NO: 10) (67C Tm) |
| Target 1 | ACAGATCTTGAGGCTCTCATGGAATGGCTAAAGACAAGACCAATCCTGTCA |
| | CCTCTGACTAAGGGGATTTTAGGATTTGTGTTCACGTTCACCGIGCCCAGT |
| | GAGCGAGGACTGCAGCGTAGACGCT (SEQ ID NO: 11) |
| Invader 1 | GAACACAAATCCTAAAATCCCCTT (SEQ ID NO: 12) |
| Activation | CCACTCTCTCTCTGATGAAACTCACGATAGAA/3SpC3/ (SEQ ID NO: |
| Probe 2-Inv | 13) |
| Flap Probe 2- | CCACTCTCTCTCTGAT (SEQ ID NO: 14) |
| Inv | |
| Fwd Primer 2 | ATCCATCTCTACTTGAAATCACAG (SEQ ID NO: 15) |
| Rev Primer 2 | AAGATCCACTTTTTTAGGCAGAT (SEQ ID NO: 16) |
| Target 2 | GATGGTTAAACTATTATAAACTTAATACTTATCCATCTCTACTTGAAATCA |
| | CAGAAAATGATTTGATTATTTTATCAGGATTGCGGTTCTATCGTGAGTTTC |
| | ATCTGCCTAAAAAAGTGGATCTTGAAATGATAATAAAT (SEQ ID NO: |
| | 17) |
| Cleavable | /56-FAM//iMe- |
| Probe-Inv | isodC/CCCTCCCTCCTCCTTCCTCCTGAGTGCCAAAA/iSpC3/AAAACC |
| | CACTCArATATCAGAGAGAGAGTGG/3SpC3/ (SEQ ID NO: 18) |

Cleavable Probes with Conversion Probes

In this example, two separate targets can be detected with a single Cleavable Probe following modification of the Flap Probes using Conversion Probes. Forward and Reverse primers 1 amplify target 1 to create an amplicon, which Activation Probe 1 reacts with, resulting in the cleavage of Activation Probe 1. The cleaved Activation Probe 1 (i.e., Flap Probe 1) can now extend along the Conversion Probe 1 since it has an extensible 3′-OH group that is complimentary to the Conversion Probe 1. After extension of Flap Probe 1 along Conversion Probe 1, the modified Flap Probe 1 can now hybridize to, and extend along Cleavable Probe 1, where it stops at the extension blocker. Because modified Flap Probe 1 extended beyond the ribobase of Cleavable Probe 1, Cleavable Probe 1 can now be cleaved at the ribobase. After the cleavable probe is cleaved, it can then denature and rehybridize to itself during the denaturation and annealing phases of the next PCR cycle. When it rehybridizes to itself, it can extend along itself, where it can attach a quencher across from the fluorophore via the dabcyl-isoGtp nucleoside quencher. This will cause a decrease in fluorescence during the reaction, and a specific melt signature during a melt analysis after PCR. A melt analysis is done by observing the signal in the reaction at more than 1 temperature.

Forward and Reverse primers 2 amplify target 2 to create an amplicon, which Activation Probe 2 reacts with to cleave Activation Probe 2. The cleaved Activation Probe 2 (i.e., Flap Probe 2) can now extend along Conversion Probe 2 to form modified Flap Probe 2. Subsequently, modified Flap Probe 2 can extend along the same Cleavable Probe 1 since it has an extensible 3′-OH group that is complimentary to the Cleavable Probe 1. This demonstrates that a single multiprobe can identify and quantify more than one target in a qPCR or digital PCR reaction.

The conversion probe may also comprise a few ribobases, such that during extension of the flap probe along the conversion probe, the polymerase adds bases opposite the ribobases, forming a cleavage site where the conversion probe is cleaved. This reduces competition in the reaction between the conversion probe and the cleavable probe for binding to the modified flap probe, in order to favor hybridization of the modified flap probe to the cleavable probe.

Universal cleavable probe reactions can be performed using 400 nM of each of the forward primer and 100 nM of the reverse primer and activation probes, 50 nM of each cleavable probe, 6.675 ng/μL cleavase 2.0 (Hologic), 1 U hotstart GoTaq polymerase (Promega), 11.2 mU RNaseH2, 20 mmol/L BTP pH 9.1, 10 mmol/L Tris pH 8.3, 50 mM KCl, 5 mmol/L MgCl₂, 0.3 mg/nL non-acetylated BSA, 1 μM Dabcyl-diGTP, 0.09 mmol/L DTT, and 250 μmol/L of each dNTP.

Cycling conditions can consist of 95° C. for 3 min; 45 cycles at 95° C. for 10 s, 54° C. for 1 s, and 57° C. for 23 s.

Primers, probes, and target sequences used for are as follows:

| Molecule | Sequence |
|---|---|
| Activation | CCTCACTCACACCTAAAATrCCCCTTAGTCAGAGGTGAC/3SpC3/ (SEQ |
| probe 1 | ID NO: 19) |
| Flap Probe 1 | CCTCACTCACACCTAAAAT (SEQ ID NO: 20) |
| Conversion | GTGCArGGCACCrGACATTTTAGGIGTGAGIGAGG (SEQ ID NO: 21) |
| Probe 1 | |
| Fwd Primer 1 | GGCTCTCATGGAATGGCTAAAGAC (SEQ ID NO: 9) (67C Tm) |
| Rev Primer 1 | ACGCTGCAGTCCTCGCTCA (SEQ ID NO: 10) (67C Tm) |
| Target 1 | ACAGATCTTGAGGCTCTCATGGAATGGCTAAAGACAAGACCAATCCTGTCA |
| | CCTCTGACTAAGGGGATTTTAGGATTTGTGTTCACGTTCACCGTGCCCAGT |
| | GAGCGAGGACTGCAGCGTAGACGCT (SEQ ID NO: 11) |
| Activation | CACCGTCGTAGATGAAACTUCACGATAGAACCGCA/3SpC3/ (SEQ ID |
| Probe 2 | NO: 22) |
| Flap Probe 2 | CACCGTCGTAGATGAAAC (SEQ ID NO: 23) |
| Conversion | GTGCArGGCACCrGACGTTTCATCTACGACGGTG (SEQ ID NO: 24) |
| Probe 2 | |
| Fwd Primer 2 | ATCCATCTCTACTTGAAATCACAG (SEQ ID NO: 15) |
| Rev Primer 2 | AAGATCCACTTTTTTAGGCAGAT (SEQ ID NO: 16) |
| Target 2 | GATGGTTAAACTATTATAAACTTAATACTTATCCATCTCTACTTGAAATCA |
| | CAGAAAATGATTTGATTATTTTATCAGGATTGCGGTTCTATCGTGAGTTTC |
| | ATCTGCCTAAAAAAGTGGATCTTGAAATGATAATAAAT (SEQ ID NO: |
| | 17) |
| Cleavable | /56-FAM//iMe- |
| Probe | isodC/CCCTCCCTCCTCCTTCCTCCTGAGGGCCAAAA/iSpC3/AAAACT |
| | CCCTCArAGTGCAGGCACCGAC/3SpC3/ (SEQ ID NO: 25) |

Cleavable Probes with 5′ Nuclease Cleavage of the Activation Probe

The following experiment will be performed to demonstrate cleavage of an activation probe using the 5′ exonuclease activity of a polymerase in the presence of a target nucleic acid during PCR, and subsequent hybridization to a cleavable probe, followed by extension to generate a cleavage site in the cleavable probe. This is expected to result in cleavage and extension of the cleavable probe to form a detectable hairpin probe. Melt analysis will be performed after the PCR reaction.

Activation and cleavable probe reactions can be performed using 400 nM of each of the primers and detection probes, 50 nM of each cleavable probe, 1 U hotstart GoTaq polymerase (Promega), 11.2 mU RNaseH2, 20 mmol/L BTP pH 9.1, 10 mmol/L Tris pH 8.3, 50 mM KCl, 5 mmol/L MgCl₂, 0.3 mg/nL non-acetylated BSA, 1 μM Dabcyl-diGTP, 0.09 mmol/L DTT, and 250 μmol/L of each dNTP.

Cycling conditions may consist of 95° C. for 3 min; 10 cycles at 95° C. for 20 s, 67° C. for 30 s, and 70° C. for 30 s; 37 cycles at 95° C. for 20 s, 53° C. for 1 min, and 70° C. for 30 s; and 40° C. hold for 30 s.

Primers, probes, and target sequences used for are as follows:

| Molecule | Sequence |
|---|---|
| Activation | CCACTCTCTCTCTGATAGTCAGAGGTGAC/3SpC3/ (SEQ ID NO: 7) |
| probe 1 | |
| Flap Probe 1 | CCACTCTCTCTCTGAT (SEQ ID NO: 8) |
| Fwd Primer 1 | GGCTCTCATGGAATGGCTAAAGAC (SEQ ID NO: 9) (67C Tm) |
| Rev Primer 1 | ACGCTGCAGTCCTCGCTCA (SEQ ID NO: 10) (67C Tm) |
| Target 1 | ACAGATCTTGAGGCTCTCATGGAATGGCTAAAGACAAGACCAATCCTGTCA |
| | CCTCTGACTAAGGGGATTTTAGGATTTGTGTTCACGTTCACCGTGCCCAGT |
| | GAGCGAGGACTGCAGCGTAGACGCT (SEQ ID NO: 11) |
| Cleavable | /56-FAM//iMe- |
| Probe | isodC/CCCTCCCTCCTCCTTCCTCCTGAGTGCCAAAA/SpC3/AAAACC |
| | CACTCArATATCAGAGAGAGAGTGG/3SpC3/ (SEQ ID NO: 18) |

### Cleavable Probes for Restriction Endonuclease Cleavage

The following example demonstrates cleavage of a ribonucleotide-containing activation probe in the presence of a target nucleic acid during PCR, and subsequent hybridization to a cleavable probe, followed by extension to generate a restriction endonuclease enzyme recognition site in the cleavable probe. The recognition site for the BstB1 nicking enzyme is 5′TT*CGAA where * marks the cleavage site. And the reverse strand is 3′AAGC*TT. This is expected to result in cleavage and extension of the cleavable probe to form a detectable hairpin probe. Melt analysis will be performed after the PCRreaction. This reaction may be used with or without 300 μM Sp-dATP-α-S alpha thiol modified dNTP. A double stranded break will occur without the phosphorothioate (PTO) linkage, and a single stranded break on the cleavable probe 01bres will occur with the extended PTO strand.

| | | Y537S | WT | RMP-01 |
|---|---|---|---|---|
| Oligo | Sequence | T_{M} | T_{M} | T_{M} |
| Activation | CTTCTTCCTTGGAACTCTr [symbol] TGACCTGCTGC/3 | 62.5 | 52.7 | 57.2 |
| probe 1nic | SpC/ (SEQ ID NO: 1) | | | |
| Cleavable | /FAM//isoC/TATATATATATATATAATGGAC | | | |
| Probe- | TCCAAAAAAAACC/iSpC3/GGTCCCATTGCAA | | | |
| 01Bres | aaaAGAGTTCCAAGGAAGAAGGG/3SpC3/ | | | |
| | (SEQ ID NO: 26) | | | |

Primers and target sequences used for PCR amplification are as follows:

| Oligo/gblock | Sequence |
|---|---|
| JET401 | TGG GCG TCC AGC ATC TC (SEQ ID NO: 4) |
| LH1375 | CAA AGG CAT GGA GCA TCT GTA (SEQ ID NO: 5) |
| TW661 | TTCTGTGTCTTCCCACCTACAGTAACAAAGGCATGGAGCATCTGTACAGCA |
| | TGAAGTGCAAGAACGTGGTGCCCCTCTCTGACCTGCTGCTGGAGATGCTGG |
| | ACGCCCACCGCCTACATGCGCCCACTAGCCGTGGAGGGGCATCCGTGGAGG |
| | AGACGGACCAAAGC (SEQ ID NO: 6) |

The amplification mixture may contain 10 mM Tris, 20 mM Bis-tris propane (BTP), 300 μg/mL BSA, 0.09 mM DTT, 2.5 mM MgCl₂, 50 mM KCl, 0.1 mM dNTPs, 1 μM Dabcyl-diGTP, 1×Titanium Taq Polymerase (Takara Biosciences), 16.2 mU/μL RNase HII (Takara Biosciences), 12U BstB1, 100 nM of JET401 oligonucleotide primer, 400 nM LH1375 oligonucleotide primer, and 50 nM of the Fam-labeled Cleavable Probe-01Bnic. Activation Probe 1nic oligos, are at a concentration of 50 nM. The target nucleic acid (TW661) was spiked into the reaction at 10,000 copies or not at all in the NTC (no template control).

Thermal cycling may be performed on an ABI 7500 Fast system (Thermo Fisher) at a final volume of 25 uL with the following conditions: 1 cycle of 2 min 20 s at 95° C.; 60 cycles of the following: 10 s at 95° C., 20 s at 58° C.

Measurements of the signal in the reaction will be taken during PCR at the 58° C. stage of each cycle and after PCR at 0.5° C. increments starting at 60° C. and end at 95° C.

The reaction mixtures containing target, cleavable probe, and activation probe are expected to result in a decrease in FAM signal during PCR and continuing to decrease throughout the PCR. Melt analysis performed after PCR is expected to show a positive melt peak with a T_{M} (melting temperature) of 64° C. The FAM signal at 60° C. is expected to be significantly less than the FAM signal at 68° C. for this condition.

Other conditions that do not have target present, or do not have activation probe present would not be expected to exhibit a change in signal during PCR, would not have a positive melt peak, and would not show a change in signal between 60° C. and 68° C.

### Dark Cleavable Probes

A comparison of the signal:noise ratio in a multiplex reaction using unquenched cleavable probes that show maximal fluorescence in the absence of target nucleic acid versus cleavable probes whose fluorescence is quenched in the absence of target nucleic acid (also referred to as dark cleavable probes) was made. Target nucleic acid and cleavable probes were added to a reaction mixture suitable for performing digital PCR and partitioning was performed on an absolute Q digital PCR system (Combinati) having 20,000 partitions per well. Signals were detected using the Combinati Q system.

In order to ascertain the signal:noise improvement provided by a dark cleavable probe population specific for a particular target nucleic acid against various concentrations of different unquenched cleavable probes populations whose targets are not present in the reaction, the dark cleavable probes were present at a concentration of 300 nM, while the different unquenched cleavable probes whose targets were not present in the reaction were included at varying concentrations. Dark cleavable probes and unquenched cleavable probes were each compared in reactions comprising cognate target nucleic acid in a background of dark cleavable probes and unquenched cleavable probes for which no cognate target nucleic acid was present. The relative concentrations of target nucleic acid specific probes to non-target specific probes were as follows:

| Probe | Target-specific Probe | Non-target specific | |
|---|---|---|---|
| concentrations | concentration | Probe concentration | |
| Expt. 1 | 300 nM | 600 | nM |
| Expt. 2 | 300 nM | 900 | nM |
| Expt. 3 | 300 nM | 1200 | nM |

Asymmetric amplification was performed using 400 nM excess primer and 100 nM limiting primer, 300 nM dark target-specific cleavable probes or 300 nM unquenched target-specific cleavable probes, and the indicated concentrations of dark or unquenched non-target specific probes, 2 U hotstart TiTaq polymerase (Takara), 64 mU RNaseHII, 10 mmol/L Bis-tris propane (BTP) pH 9.1, 10 mmol/L BTP pH 8.0, 10 mmol/L Tris pH 8.3, 50 mM KCl, 2.5 mmol/L MgCl₂, 0.3 mg/nL non-acetylated Bovine Serum Albumin, 1 μM Dabcyl-diGTP, 0.09 mmol/L dithiothreitol, and 250 p mol/L of each dNTP.

The sequences of the primers, probes, and target nucleic acid used in the reaction were as follows:

| Molecule | Sequence |
|---|---|
| unquenched target- | /56-FAM//iMe- |
| specific cleavable | isodC/CCTCCTCCTCCTTCCTCCTGAGGGCCAAAA/iSpC3/AAAA |
| probe, JB0041 | CCCCCTCArATGACCTGCTG/3SpC3/ (SEQ ID NO: 27) |
| dark target specific | /BHQ1/TCCTrCCTCC/T-FAM//iMe- |
| cleavable probe, | isodC/CCTCCTCCTCCTTCCTCTGAGGGCCAAAA/iSpC3/AAAAC |
| JT0393 | CCCCTCArATGACCTGCTG/3Spc3/ (SEQ ID NO: 29) |
| non-target specific | /56-FAM//iMe- |
| cleavable probe, | isodC/ATTTATATATTATTTAAGGGACACCAAAA/iSpC3/AAACC |
| JT0363 | TGTCCCrCCTATGACCTGCT/3SpC3/ (SEQ ID NO: 28) |
| dark non-target | /BHQ1/TCCTrCCTCT/T- |
| specific cleavable | FAM//iMeisodC/TATATATTATTTAAGGGACACCAAAA/iSpC3/ |
| probe, JT0411 | AAACCTGTCCCrCCTATGACCTGCT/3SpC3/ (SEQ ID NO: |
| | 30) |
| target nucleic acid | TTTGGCCAAGCCCGCTCATGATCAAACGCTCTAAGAAGAACAGCCTG |
| | GCCTTGTCCCTGACGGCCGACCAGATGGTCAGTGCCTTGTTGGATGC |
| | TGAGCCCCCCATACTCTATTCCGAGTATGATCCTACCAGACCCTTCA |
| | GTGAAGCTTCGATGATGGGCTTACTGACCAACCTGGCAGACAGGGAG |
| | CTGGTTCACATGATCAACTGGGCGAAGAGGGIGCCAGGCTTTGTGGA |
| | TTTGACCCTCCATGATCAGGTCCACCTTCTAGAATGTGCCTGGCTAG |
| | AGATCCTGATGATTGGTCTCGTCTGGCGCTCCATGGAGCACCCAGGG |
| | AAGCTACTGTTTGCTCCTAACTTGCTCTTGGACAGGAACCAGGGAAA |
| | ATGTGTAGAGGGCATGGTGGAGATCTTCGACATGCTGCTGGCTACAT |
| | CTCAAATCTATTATTTTGCTTAATTCTGGAGTGTACACATTTCTGTC |
| | CAGCACCCTGAAGTCTCTGGAAGAGAAGGACCATATCCACCGAGTCC |
| | TGGACAAGATCACAGACACTTTGATCCACCTGATGGCCAAGGCAGGC |
| | CTGACCCTGCAGCAGCAGCACCAGCGGCTGGCCCAGCTCCTCCTCAT |
| | CCTCTCCCACATCAGGCACATGAGTAACAAAGGCATGGAGCATCTGT |
| | ACAGCATGAAGTGCAAGAACGTGGTGCCCCTCAATGACCTGCTGCTG |
| | GAGATGCTGGACGCCCACCGCCTACATGCGCCCACTAGCCGTGGAGG |
| | GGCATCCGTGGAGGAGACGGACCAAAGCCACTTGGCCACTGCGGGCT |
| | CTACTTCATCGCATTCCTTGCAAAAGTATTACATCACGGGGGAGGCA |
| | GAGGGTTTCCCTGCCACGGTCTGAGAGCTCCCTGGCTCCCACACGGT |
| | TCAGATAATCCCTGCTGCATTTTACCCTCATCATGCACCACTTTAGC |
| | CAAATTCTGTCT (SEQ ID NO: 31) |
| fwd primer, JT401 | TGGGCGTCCAGCATCTC (SEQ ID NO: 4) |
| rev primer, LH1375 | CAAAGGCATGGAGCATCTGTA (SEQ ID NO: 5) |

Cycling conditions consisted of 95° C. for 3 min, 45 cycles at 95° C. for 10 s, 54° C. for 1 s, 57° C. for 23 s. Images were taken of each digital PCR reaction using the camera in the Absolute Q system at various temperatures (e.g., 55° C. and 90° C.) using the filter and LED settings appropriate for each dye. A ratio of image intensity at the two temperatures was calculated for each partition and plotted on a 1 dimensional plot. Signal to noise ratios were determined by comparing the population of negative partitions to the population of positive partitions in each reaction. The variability of each population as well as the separation of the positive and negative populations are calculated using techniques known in the art. Specifically, the RES calculation is equal to the difference between the mean of the positive and negative partitions divided by the average standard deviation of the positive and negative partitions.

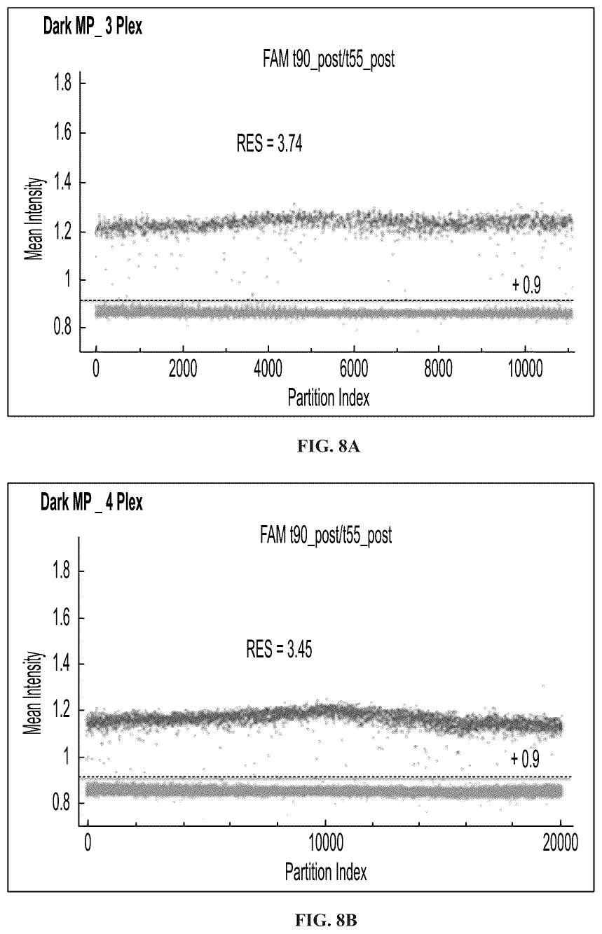

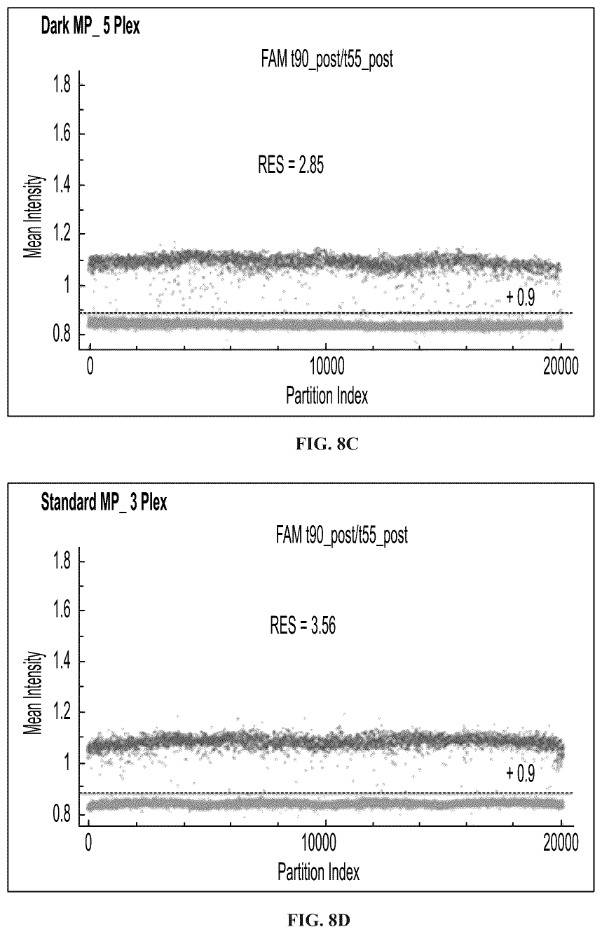

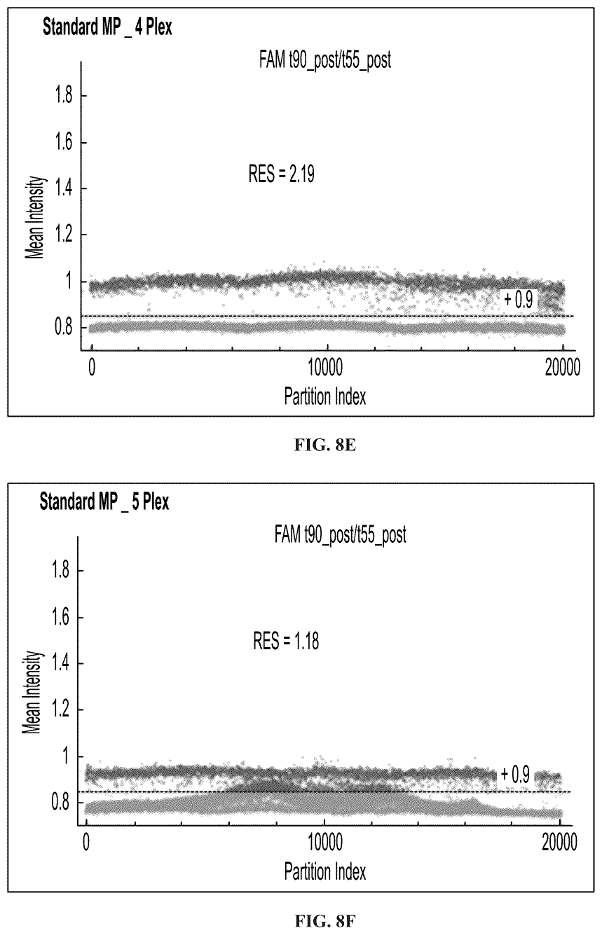

FIG. 8 illustrates that relative to unquenched cleavable probes, dark cleavable probes show an increased signal:noise separation at all concentrations of probe tested. Even in high concentrations of unreacted non-target specific probes, specific signals from target-specific dark cleavable probes can be distinguished from background fluorescence.

### Cleavable Probes Using an Activation Probe Triggered by Exonuclease Cleavage

The following experiment was performed to demonstrate cleavage of an activation probe in the presence of a target nucleic acid during PCR using a DNA polymerase with exonuclease activity, and subsequent hybridization to a cleavable probe, followed by an extension to generate a cleavage site in the cleavable probe. This resulted in cleavage and extension of the cleavable probe to form a detectable hairpin probe. Melt analysis was performed after PCR.

| | | Amplicon | Cleavable |
|---|---|---|---|
| Oligo | Sequence | Tm | probe Tm |
| Activation | GAGGGAGGATATGGCCTGCTGCTGGAG | 72.5 | 44.6 |
| probe nR-1 | ATGCTGGAC/3SpC3/ (SEQ ID | | |
| | NO: 32) | | |
| Activation | GGAGGGAGGATATGGCCTGCTGCTGGA | 72.5 | 49.2 |
| probe nR-2 | GATGCTGGAC/3SpC3/ (SEQ ID | | |
| | NO: 33) | | |
| Activation | GAGGGAGGATATGrGCCTGCTGCTGG/ | 63.1 | 44.6 |
| probe R-L3 | SpC3/ (SEQ ID NO: 34) | | |
| Cleavable | /56-FAM//iMe- | n/a | n/a |
| probe L003- | isodC/ ATTTATATATTATTTAATGAA | | |
| G_ECO1.1 | CACACCAAAAAAAACC/iSpC3/TGTG | | |
| | TTCArCtctataaaCATATCCTCCCTC | | |
| | CTTCTCT/3SpC3/ (SEQ ID NO: | | |
| | 35) | | |

| Oligo/gblock | Sequence |
|---|---|
| LH1373 | GAT GCC CCT CCA CGG (SEQ ID NO: 36) |
| LH1375 | CAA AGG CAT GGA GCA TCT GTA (SEQ ID NO: 5) |
| TW660 | TTCTGTGTCTTCCCACCTACAGTAACAAAGGCATGGAGCATCTGTACAGCA |
| | TGAAGTGCAAGAACGTGGTGCCCCTCTATGGCCTGCTGCTGGAGATGCTGG |
| | ACGCCCACCGCCTACATGCGCCCACTAGCCGTGGAGGGGCATCCGTGGAGG |
| | AGACGGACCAAAGC (SEQ ID NO: 37) |

The amplification mixture contained 10 mM Tris, 20 mM Bis-tris propane (BTP), 300 μg/mL BSA, 0.09 mM DTT, 2.5 mM MgCl₂, 50 mM KCl, 0.1 mM dNTPs, 1 μM Dabcyl-diGTP, 1 U/uL Phoenix Taq polymerase (Enzymatics), 16.2 mU/μL RNase HII (Takara Biosciences), 400 nM each of the LH1375 and LH1373 oligonucleotide primers, and 200 nM of the Fam-labeled Cleavable probe L003-G_ECO1.1. Activation Probe oligos, when present, were at a concentration of 200 nM. The target nucleic acid (TW660) was spiked into the reaction at 10,000 copies or not at all in the NTC (no template control).

Thermal cycling was performed on an ABI 7500 Fast system (Thermo Fisher) at a final volume of 25 uL with the following conditions: 1 cycle of 2 min 20 s at 95° C.; 50 cycles of the following: 10 s at 95° C., 23 s at 56° C.

Measurements of the signal in the reaction were taken during PCR at the 56° C. stage of each cycle and after PCR at 0.5° C. increments starting at 60° C. and end at 95° C.

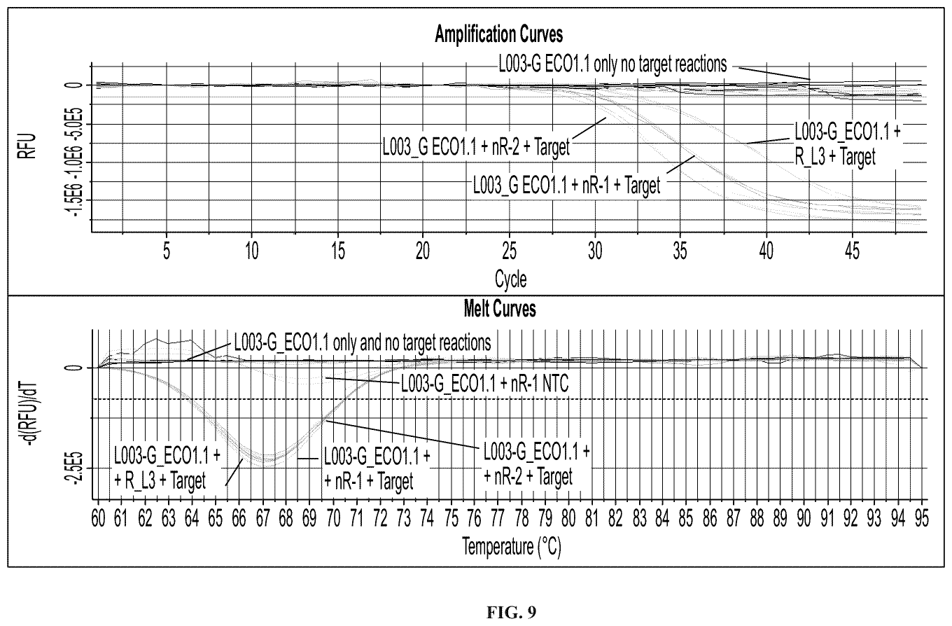

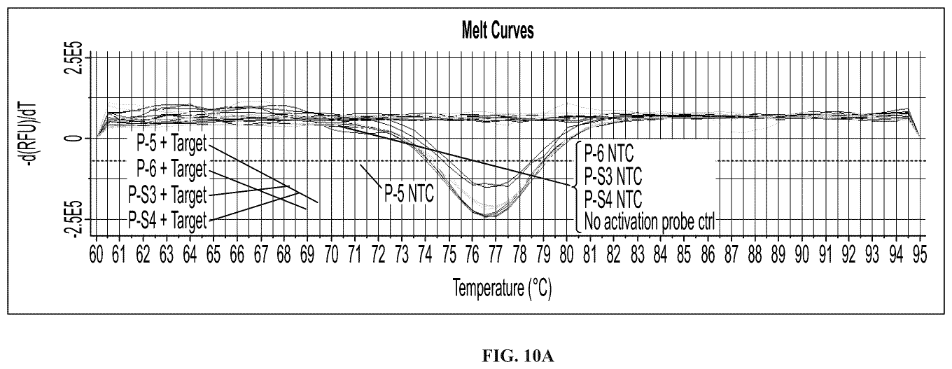

The reaction mixtures containing target, cleavable probe, and activation probe resulted in a decrease in FAM signal starting around cycle 28 during PCR and continuing to decrease throughout the PCR (FIG. 9, upper panel). Melt analysis performed after PCR showed a positive melt peak with a T_{M} (melting temperature) of 64° C. (FIG. 9, lower panel). The FAM signal at 60° C. was significantly less than the FAM signal at 68° C. for this condition.

### Cleavable Probes Using Activation Probes Containing an Internal C3 Spacer to Improve Assay Specificity

The following experiment was performed to demonstrate cleavage of an activation probe containing an internal C3 spacer in the presence of a target nucleic acid during thermal cycling, and subsequent hybridization to a cleavable probe, followed by an extension to generate a cleavage site in the cleavable probe. The presence of a C3 spacer within the activation probe sequence did not inhibit cleavage and extension of the cleavable probe to form a detectable hairpin probe. Melt analysis was performed after PCR.

| | | Amplicon | Cleavable |
|---|---|---|---|
| Oligo | Sequence | Tm | probe Tm |
| Activation | GGGaGGCATCTGrCCCAGCACCCT/ | 63.9 | 48.8 |
| probe P-5 | 3Spc3/ (SEQ ID NO: 38) | | |
| Activation | GGaGGCATCTGrCCCAGCACCCT/ | 63.9 | 43.5 |
| probe P-6 | 3SpC3/ (SEQ ID NO: 39) | | |
| Activation | GCGAaGGGaG/iSpC3/CATCTGrCCC | 63.9 | 52.6 |
| probe P-S3 | AGCACCCT/3SpC3/ | | |
| | (SEQ ID NO: 40) | | |
| Activation | GGaGGCATCTGrCCCAG/iSpC3/ | 69.6 | 43.5 |
| probe P-S4 | ACCCTGAAGTCTCTG/3SpC3/ | | |
| | (SEQ ID NO: 41) | | |
| Cleavable | /56-FAM//iMe- | n/a | Varies |
| probe | isodC/Ccctataattcctatcttcca | | |
| M001.2- | ggcTCAAAAAAAACC/iSpC3/gcctg | | |
| P_ECO1.1 | garCctttaacaCAGATGCCtCCCtTC | | |
| | Gcctc/3SpC3/ | | |
| | (SEQ ID NO: 42) | | |
| L003_D538G_ | /56-FAM//iMe- | Background | 0 |
| ECO1.1 | isodC/ATTTATATATTATTTAATGAA | probe-no | |
| | CACACCAAAAAAAACC/iSpC3/TGTG | activity | |
| | TTCArCtctataaaCATATCCTCCCTC | | |
| | CTTCTCT/3SpC3/ | | |
| | (SEQ ID NO: 43) | | |
| M001.2_ | /56-FAM//iMe-isodc/ | Background | 0 |
| S463P_ | CcctataattcctatcttccaggcTCA | probe-no | |
| ECO1.1 | AAAAAAACC/iSpC3/ | activity | |
| | gcctggarCctttaacaCAGATGCCtC | | |
| | CCtTCGcctc/3SpC3/ | | |
| | (SEQ ID NO: 44) | | |

### Standard PCR

Primers and target sequences used for PCR amplification were as follows:

| Oligo/gblock | Sequence |
|---|---|
| JET401 | TGG GCG TCC AGC ATC TC (SEQ ID NO: 4) |
| LH1375 | CAA AGG CAT GGA GCA TCT GTA (SEQ ID NO: 5) |
| TW671 | CTCTGGGTCTCCTAGACCTCATCCTCTTTGAGCTTCTCTCTCTCACTCTCT |
| | CTCTGCGCATTCAGGAGTGTACACATTTCTGCCCAGCACCCTGAAGTCTCT |
| | GGAAGAGAAGGACCATATCCACCGAGTCCTGGACAAGATCACAGACACTTT |
| | GATCCACCTGATGG (SEQ ID NO: 45) |

The amplification mixture for standard PCR contained 10 mM Tris, 20 mM Bis-tris propane (BTP), 300 μg/mL BSA, 0.09 mM DTT, 2.5 mM MgCl₂, 50 mM KCl, 0.1 mM dNTPs, 1 μM Dabcyl-diGTP, 2×Titanium Taq Polymerase (Takara Biosciences), 16.2 mU/μL RNase HII (Takara Biosciences), 100 nM of JET401 oligonucleotide primer, 400 nM LH1375 oligonucleotide primer, and 200 nM of the Fam-labeled Cleavable probe M001.2-P_ECO1.1. Activation Probe oligos, when present, were at a concentration of 200 nM. The target nucleic acid (TW671) was spiked into the reaction at 10,000 copies or not at all in the NTC (no template control).

Thermal cycling was performed on an ABI 7500 Fast system (Thermo Fisher) at a final volume of 25 uL with the following conditions: 1 cycle of 2 min 20 s at 95° C.; 50 cycles of the following: 10 s at 95° C., 23 s at 56° C. Measurements of the signal in the reaction were taken during PCR at the 56° C. stage of each cycle and after PCR at 0.5° C. increments starting at 60° C. and end at 95° C. The presence or absence of a melt signal was used to evaluate reaction positivity.

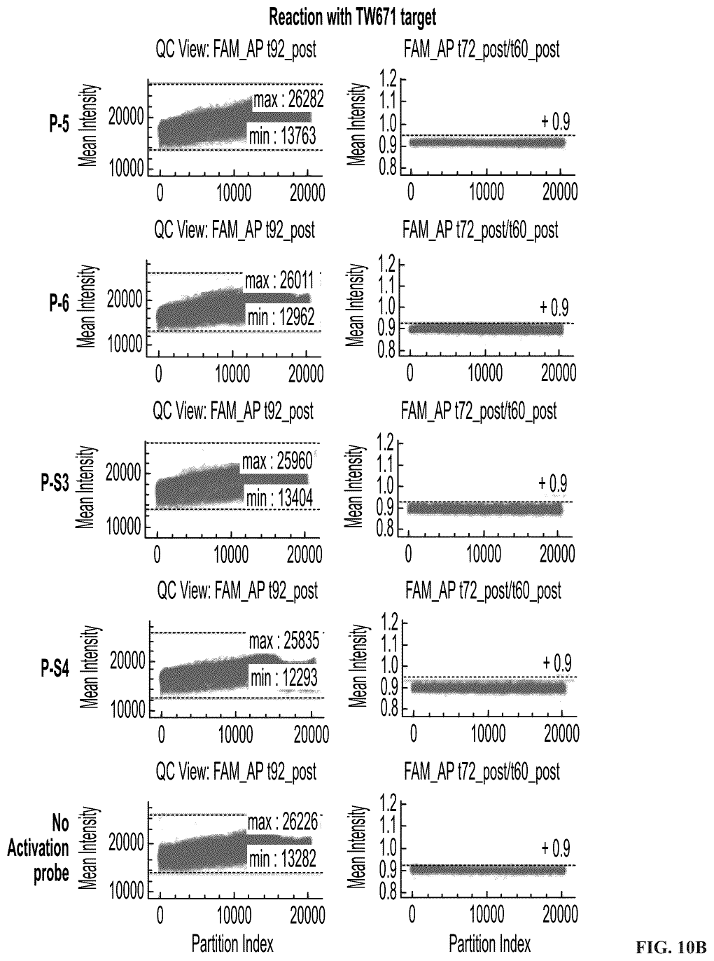

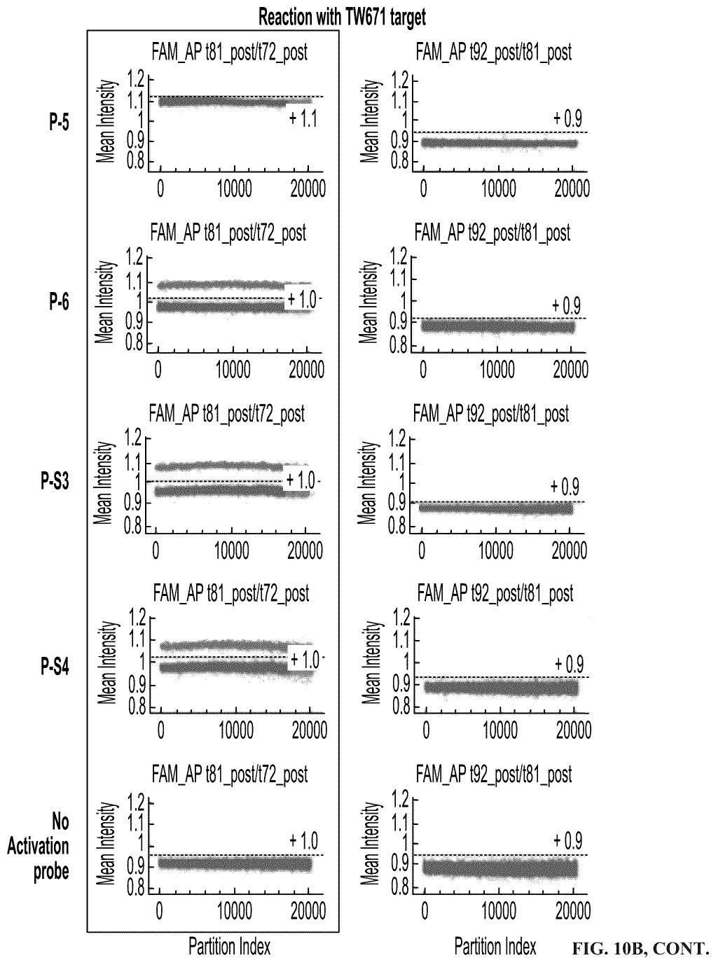

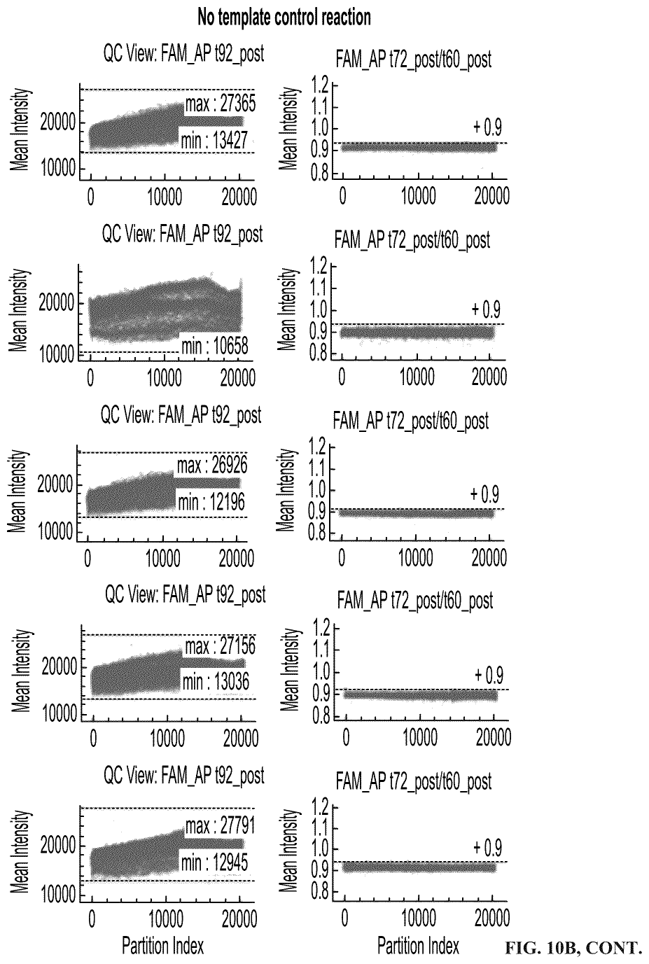

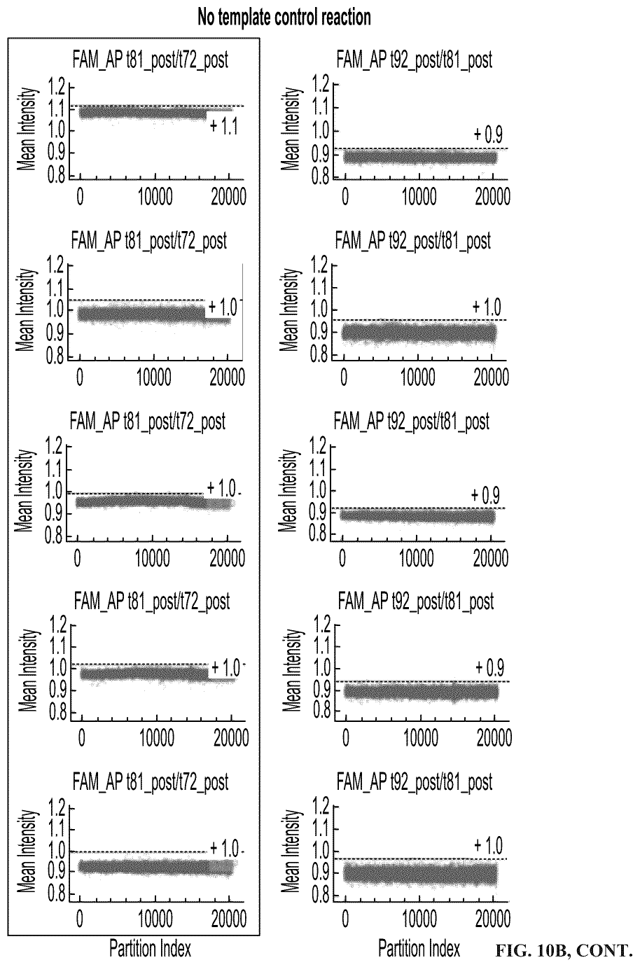

In real-time PCR (FIG. 10A), the reaction mixture containing 200 nM Cleavable probe M001.2-P_ECO1.1 and 200 nM Activation probes P-6, P-S3, or P-S4 resulted in a target-specific melt at 76.7 C. The presence of Activation probe P-5 resulted in non-specific signal in the absence of target. In the absence of any Activation probe, no non-specific melt was observed.

### Digital PCR

The amplification mixture for digital PCR contained 10 mM Tris, 20 mM Bis-tris propane (BTP), 300 μg/mL BSA, 0.09 mM DTT, 2.5 mM MgCl₂, 50 mM KCl, 0.1 mM dNTPs, 1 μM Dabcyl-diGTP, 2×Titanium Taq Polymerase (Takara Biosciences), 44.8 mU/μL RNase HII (Takara Biosciences), 100 nM of JET401 oligonucleotide primer, 400 nM LH1375 oligonucleotide primer, 200 nM of the Fam-labeled Cleavable probe M001.2-P_ECO1.1, 200 nM each of the Fam-labeled cleavable probes L003_D538G_ECO1.1 and M001.2_S463P_ECO1.1 as signal background. Activation Probe 1 oligos were at a concentration of 200 nM. The target nucleic acid (TW671) was spiked into the reaction at approximately 3,000 copies or not at all in the NTC (no template control).

Digitization and thermal cycling were performed on an Applied Biosystems QuantStudio Absolute Q Digital PCR System. 9 μL of reaction mixture was transferred to the well of a MAP16 consumable and covered with 15 μL of Isolation Buffer (Applied Biosystems). Gaskets were appended to each well and the consumable was transferred to the dPCR system. The consumable was pressurized at 75 psi for 25 min and held at 50 psi to load and digitize the sample across approximately 20,000 partitions. Amplification was performed by pre-heating the consumable at 96° C. for 10 min., followed by 49 cycles of the following: 5 s at 93° C., 20 s at 60° C.; pressure was maintained at 50 psi throughout the PCR reaction. A 10 min isothermal hold at 55° C. was incorporated after PCR, but before imaging. After thermal cycling, Combinati analysis software was used to locate and identify partitions within the unit and to acquire images of partitions at 60° C. (T1), 72° C. (T2), 81° C. (T3), and 92° C. (T4) with optical filters set to preferentially obtain fluorescence values for the FAM-AP fluorophore. Ratios were calculated for fluorescence signals measured at successive temperatures and plotted on a 1D amplitude plot. The detectable hairpin probe formed from Cleavable Probe M001.2-P_ECO1.1 after cleavage has a T_{M} of 76.5° C. and was thus expected to show maximum change in fluorescence (ratio >1) between T2 and T3. Cleavable probes L003_D538G_ECO1.1 and M001.2_S463P_ECO1.1, included herein as background signal to demonstrate assay specificity in a complex signal matrix, have post-cleavage melts of approximately 66 C and 87 C and would be expected to show maximum change in fluorescence (ratio >1) between T1 and T2 or T4 and T3, respectively if they were to be activated.

In digital PCR (FIG. 10B), the reaction mixture containing 200 nM Cleavable probe M001.2-P_ECO1.1 and 200 nM Activation probes P-6, P-S3, or P-S4 resulted in a decrease in FAM signal between T2 (72° C.) and T3 (81° C.), resulting in a clear positive population in the 1D amplitude plot (FIG. 10B, left panel). Activation probe P-5 resulted in non-specific background cleavage of Cleavable probe M001.2-P_ECO1.1, and no positive population could be observed in the presence of target. In conditions where target was not present, there was no change in signal from T2 to T3, and no positive partitions were detected (FIG. 10B, right panel) No signal change was noted for either of the background signal probes.

1. A method for detecting a first target nucleic acid in a sample, the method comprising the steps of:
a) contacting the sample with reagents comprising:
i) a first activation probe comprising from 5′ to 3′, a first region that is not complementary to the first target nucleic acid, a second region comprising a first cleavage region, wherein the second region is complementary to the first target nucleic acid, wherein the first cleavage region is capable of forming a first cleavage site when double stranded;
ii) a first cleavable probe comprising from 5′ to 3′, a first region comprising a label, a second region, a loop region comprising a 3′ extension blocker at the 3′ end, a third region that is complementary to at least a portion of the second region, and a fourth region comprising a first cleavage region at the 5′ end, wherein at least a portion of the fourth region 3′ of the first cleavage region is complementary to the first region of the first activation probe, wherein the first cleavage region is capable of forming a first cleavage site when double stranded; and
iii) one or more nucleases capable of cleaving the first cleavage site of the first activation probe and the first cleavage site of the first cleavable probe;
b) performing a reaction to form a first hairpin probe having a first T_{M} if any first target nucleic acid is present in the sample, wherein the reaction includes the steps of:
i) specifically hybridizing the second region of the first activation probe to any first target nucleic acid, thereby forming a first cleavage site at the first cleavage region of the first activation probe;
ii) cleaving the first cleavage site at the first cleavage region of the activation probe to release a first flap probe, comprising the first region of the first activation probe and at least one nucleotide of the second region of the first activation probe, from the hybridized first activation probe;
iii) hybridizing the first flap probe to the fourth region of the first cleavable probe;
iv) extending the hybridized first flap probe past the first cleavage region of the first cleavable probe using the first cleavable probe as a template, thereby forming a first cleavage site at the first cleavage region of the first cleavable probe;
v) cleaving the first cleavage site at the first cleavage region of the first cleavable probe to form a first truncated probe; and
vi) hybridizing the second and third regions of the first truncated probe and extending the hybridized third region to form a first hairpin probe having a first T_{M}; and
c) detecting the presence of the first target nucleic acid by detecting the presence of the first hairpin probe.

2. The method of claim 1, wherein step b) further comprises performing an amplification reaction to amplify any first target nucleic acid present in the sample.

3. The method of claim 1, wherein the label is a first member of a reporter-quencher pair and extension of the hybridized third region to form the first hairpin probe results in incorporation of the second member of the reporter-quencher pair at a location that permits interaction of the first and second members of the reporter-quencher pair.

4. The method of claim 3, wherein the first and second members of the reporter-quencher pair are each coupled to complementary non-natural bases respectively.

5. The method of claim 4, wherein the non-natural bases are isoC and isoG.

6. The method of claim 1, wherein the label comprises a reporter-quencher pair arranged such that the quencher quenches the reporter signal when the first region of the first cleavable probe is single stranded and extension of the hybridized third region separates the reporter and quencher to release the reporter from quenching.

7. The method of claim 1, wherein the hairpin probe is detected by performing melt analysis.

8. The method of claim 3, wherein the hairpin probe is detected by detecting signals from the reporter at at least one temperature below the first T_{M} and at at least one temperature above the first T_{M} and detecting the presence of the target nucleic acid when a difference between the signal detected at the at least one temperature below the first T_{M} and the signal detected at the at least one temperature above the first T_{M} is detected.

9. The method of claim 1, wherein the first activation probe is cleaved by an invader assay cleavage event.

10. The method of claim 1, wherein the first cleavage region of the first activation probe comprises at least one ribonucleotide and the first activation probe is cleaved by an endoribonuclease.

11. The method of claim 10, wherein the endoribonuclease is RNase HII.

12. The method of claim 2, wherein the first activation probe is cleaved by a polymerase having 5′ nuclease activity.

13. The method of claim 1, wherein the fourth region of the first cleavable probe comprises one or more ribonucleotides and cleavage of the first cleavage region of the first cleavable probe is performed by RNase HII.

14. The method of claim 1, wherein cleavage of the first cleavable probe is performed by a restriction enzyme or a nicking enzyme.

15. The method of claim 1, further comprising detecting the presence of a second target nucleic acid wherein the method further comprises the steps of:
d) contacting the sample with reagents comprising:
i) a second activation probe comprising from 5′ to 3′, a first region that is not complementary to the first or second target nucleic acids, a second region comprising a first cleavage region, wherein the second region is complementary to the second target nucleic acid, wherein the first cleavage region is capable of forming a first cleavage site when double stranded;
ii) a second cleavable probe comprising from 5′ to 3′, a first region comprising a label, a second region, a loop region comprising a 3′ extension blocker at the 3′ end, a third region that is complementary to at least a portion of the second region, and a fourth region comprising a first cleavage region at the 5′ end, wherein at least a portion of the fourth region 3′ of the first cleavage region is complementary to the first region of the second activation probe, wherein the first cleavage region is capable of forming a first cleavage site when double stranded; and
iii) one or more nucleases capable of cleaving the first cleavage site of the second activation probe and the first cleavage site of the second cleavable probe;
e) performing a reaction to form a second hairpin probe having a second T_{M} if any second target nucleic acid is present in the sample, wherein the reaction includes the steps of:
i) specifically hybridizing the second region of the second activation probe to any second target nucleic acid, thereby forming a first cleavage site at the first cleavage region of the second activation probe;
ii) cleaving the first cleavage site of the second activation probe to release a second flap probe, comprising the first region of the second activation probe and optionally between one and four nucleotides of the second region of the second activation probe, from the hybridized second activation probe;
iii) hybridizing the second flap probe to the fourth region of the second cleavable probe;
iv) extending the hybridized second flap probe past the first cleavage region of the second cleavable probe using the second cleavable probe as a template, thereby forming a first cleavage site at the first cleavage region of the second cleavable probe;
v) cleaving the first cleavage site of the second cleavable probe to form a second truncated probe;
vi) hybridizing the second and third regions of the second truncated probe and extending the hybridized third region to form a second hairpin probe having a second T_{M} that is different than the first T_{M} of the first hairpin probe; and
f) detecting the presence of the second target nucleic acid by detecting the presence of the second hairpin probe.

16. The method of claim 15, wherein step b) further comprises performing an amplification reaction to amplify any second target nucleic acid present in the sample.

17. The method of claim 15, wherein the labels on the first and second cleavable probes comprise the same reporter and the first T_{M} and second T_{M} are at least 5 degrees Celsius different, and wherein the first and second hairpins are detected by performing the steps of:
g) detecting signal from the reporter at a first temperature that is below the first T_{M} and at a second temperature that is above the first T_{M};
h) detecting signal from the reporter at a third temperature that is below the second T_{M} and a fourth temperature that is above the second T_{M}, wherein the third temperature is equal to or greater than the second temperature; and
i) detecting the presence of the first target nucleic acid when there is a difference between signals detected at the first and second temperatures, and detecting the presence of the second target nucleic acid when there is a difference between signals detected at the third and fourth temperatures.

18. The method of claim 1, further comprising detecting the presence of a second target nucleic acid in the sample wherein the method further comprises the steps of:
d) contacting the sample with reagents comprising:
i) a second activation probe comprising from 5′ to 3′, a first region that is not complementary to the first or second target nucleic acids and has the same sequence as the first region of the first activation probe, and a second region comprising a first cleavage region, wherein the second region is complementary to the second target nucleic acid, wherein the first cleavage region is capable of forming a first cleavage site when double stranded; and
ii) one or more nucleases capable of cleaving the first cleavage site of the second activation probe;
e) performing a reaction to form the first hairpin probe having the first T_{M} if any second target nucleic acid is present in the sample, wherein the amplification reaction includes the steps of:
i) specifically hybridizing the second region of the second activation probe to the second target nucleic acid, thereby forming a first cleavage site at the first cleavage region of the second activation probe;
ii) cleaving the first cleavage site of the second activation probe to release a second flap probe, comprising the first region of the second activation probe and at least one nucleotide of the second region of the second activation probe, from the hybridized second activation probe;
iii) hybridizing the second flap probe to the fourth region of the first cleavable probe;
iv) extending the hybridized second flap probe past the first cleavage site of the first cleavable probe using the first cleavable probe as a template, thereby forming a first cleavage site at the first cleavage region of the first cleavable probe;
v) cleaving the first cleavage site of the first cleavable probe to form the first truncated probe;
vi) hybridizing the second and third regions of the first truncated probe and extending the hybridized third region to form the first hairpin probe having the first T_{M}; and
f) detecting the presence of the first or second target nucleic acid by detecting the presence of the first hairpin probe.

19. The method of claim 18, wherein step b) further comprises performing an amplification reaction to amplify any second target nucleic acid present in the sample.

20. The method of claim 1, wherein the method comprises detecting the presence of the first target nucleic acid or a second distinct target nucleic acid in the sample by detecting the presence of the first hairpin probe, wherein the reagents contacting the sample include a second activation probe having a 5′ first region that is not complementary to the first or second target nucleic acid and is the same as the 5′ first region of the first activation probe, and a second region comprising a first cleavage site, wherein the second region of the second activation probe is complementary to the second target nucleic acid.

21. A method for detecting a first target nucleic acid in a sample, the method comprising the steps of:
a) contacting the sample with reagents comprising:
i) a first activation probe comprising from 5′ to 3′, a first region that is not complementary to the first target nucleic acid, a second region comprising a first cleavage region, wherein the second region is complementary to the first target nucleic acid, wherein the first cleavage region is capable of forming a first cleavage site when double stranded;
ii) a first cleavable probe comprising from 5′ to 3′, a first region comprising a label, a second region, a loop region comprising a 3′ extension blocker at the 3′ end, a third region that is complementary to at least a portion of the second region, and a fourth region comprising a first cleavage region at the 5′ end, wherein the fourth region 3′ of the cleavage site is not complementary to the first region of the first activation probe, wherein the first cleavage region is capable of forming a first cleavage site when double stranded;
iii) a conversion probe comprising from 5′ to 3′, a first region that has the same sequence as at least a portion of the fourth region of the first cleavable probe and a second region that is complementary to the released first region of the first activation probe; and
iv) one or more nucleases capable of cleaving the first cleavage site of the first activation probe and the first cleavage site of the first cleavable probe;
b) performing a reaction to form a first hairpin probe having a first T_{M} if any first target nucleic acid is present in the sample, wherein the reaction includes the steps of:
i) specifically hybridizing the second region of the first activation probe to any first target nucleic acid, thereby forming a first cleavage site at the first cleavage region of the first activation probe;
ii) cleaving the first cleavage site at the first cleavage region of the activation probe to release a first flap probe, comprising the first region of the first activation probe and at least one nucleotide of the second region of the first activation probe, from the hybridized first activation probe;
iii) hybridizing the released first flap probe to the conversion probe;
iv) extending the hybridized first flap probe using the conversion probe as a template to form a modified first flap probe having a 3′ end that is complementary to the fourth region of the first cleavable probe;
v) denaturing the modified first flap probe from the conversion probe for subsequent hybridization to the fourth region of the first cleavable probe
vi) hybridizing the modified first flap probe to the fourth region of the first cleavable probe;
vii) extending the hybridized modified first flap probe past the first cleavage region of the first cleavable probe using the first cleavable probe as a template, thereby forming a first cleavage site at the first cleavage region of the first cleavable probe;
viii) cleaving the first cleavage site at the first cleavage region of the first cleavable probe to form a first truncated probe;
ix) hybridizing the second and third regions of the first truncated probe and extending the hybridized third region to form a first hairpin probe having a first T_{M}; and
c) detecting the presence of the first target nucleic acid by detecting the presence of the first hairpin probe.

22-29. (canceled)

30. A first cleavable probe for detecting a target nucleic acid, the probe comprising from 5′ to 3′, a first region comprising a label, a second region, a loop region comprising a 3′ extension blocker at the 3′ end, a third region that is complementary to at least a portion of the second region, and a fourth region comprising a first cleavage site at the 5′ end, wherein at least a portion of the fourth region 3′ of the cleavage site is complementary to a first region of a first activation probe, wherein the first activation probe comprises from 5′ to 3′, a first region that is not complementary to the target nucleic acid, a second region comprising a first cleavage site, wherein the second region is complementary to the target nucleic acid to be detected.

31-42. (canceled)

43. A method for detecting a first target nucleic acid in a sample, the method comprising the steps of:
a) contacting the sample with reagents comprising:
i) a first cleavable probe comprising, from 5′ to 3′, a first region comprising a quencher positioned 5′ to a first cleavage region, a second region comprising a reporter labeled non-natural nucleotide, a third region comprising a 3′ extension blocker at the 3′ end, a fourth region that is complementary to at least a portion of the third region, a second cleavage region, and a fifth region, wherein at least a portion of the first cleavable probe is complementary to the first target nucleic acid, wherein the portion of the first cleavable probe that is complementary to the first target nucleic acid spans the 3′ end of the fourth region, the second cleavage region, and the 5′ end of the fifth region;
ii) one or more nucleases capable of cleaving the first cleavage site of the first cleavable probe and the second cleavage site of the second cleavable probe;
b) performing a reaction to form a first hairpin probe having a first T_{M} if any first target nucleic acid is present in the sample, wherein the reaction includes the steps of:
i) specifically hybridizing the first target nucleic acid to the first cleavable probe, thereby forming a cleavage site at the second cleavage region of the first cleavable probe;
ii) cleaving the first cleavable probe at the cleavage site formed at the second cleavage region to form a first truncated probe;
iii) hybridizing the third and fourth regions of the first truncated probe and extending the hybridized fourth region to form a hairpin probe having a first T_{M} and a quencher labeled non-natural nucleotide positioned opposite the reporter labeled non-natural nucleotide of the second region of the first cleavable probe and forming a cleavage site at the first cleavage region of the first cleavable probe;
iv) cleaving the first cleavable probe at the cleavage site formed at the first cleavage region to release the first quencher from the first cleavable probe;
c) detecting the presence of the target nucleic acid by detecting the presence of the hairpin probe.

44-52. (canceled)
